(19) United States
(12) Patent Application Publication
(10) Pub. No.: US 2026/0209310 A1
(43) Pub. Date: Jul. 23, 2026

(54) COMPOSITION AND USE THEREOF FOR THE TREATMENT OF HEREDITARY DISEASES

(71) Applicant: QUIDDITAS SA, Liège (Sart-Tilman) (BE)

(72) Inventors: François CHERBONNEAU, Marche-en-Famenne (BE); Aurore CHERBONNEAU PRUNEVIEILLE, Marche-en-Famenne (BE)

(73) Assignee: QUIDDITAS SA, Liège (Sart-Tilman) (BE)

(21) Appl. No.: 19/143,616

(22) PCT Filed: Dec. 28, 2023
(86) PCT No.: PCT/EP2023/087872

§ 371 Date: Jun. 26, 2025

(30) Foreign Application Priority Data
Dec. 28, 2022 (BE) 20226100

(51) Int. Cl. C07K 14/715; C12N 5/0789; C12N 15/11; C12N 15/85; C12N 15/90

(52) U.S. Cl. CPC C07K 14/7155; C12N 5/0647; C12N 15/111; C12N 15/85; C12N 15/907; C12N 2510/00; C12N 2800/90

(57) ABSTRACT

A composition having a first single-stranded nucleic acid molecule with a region complementary to a target and transposase recognition sites, a second single-stranded nucleic acid molecule with a region complementary to a target and transposase recognition sites, and a third single-stranded molecule having a sequence coding for a portion of the interleukin 2 gamma receptor (IL2-Rγ), or coding for the complete IL2-Rγ receptor and transposase binding sites. Also, the use of this composition to replace the IL2Rγ gene in treating hereditary diseases, such as X-linked Severe Combined Immunodeficiency (SCID-X) caused by mutations in the IL2Rγ gene.

### FIELD

The invention relates to a composition and the use thereof for the treatment of hereditary diseases.

### BACKGROUND

X-linked Severe Combined Immune Deficiency (SCID-X) is caused by mutations in the IL2Rγ gene, which induce immune system dysfunction leading to severe infections, fevers, and rashes.

The gamma subunit of the interleukin-2 receptor (IL2-Rγ), regulated by the IL2-Rγ gene, is common to many interleukin receptors (IL-2, IL-4, IL-7, IL-9, IL-15) and is present on the surface of hematopoietic stem cells. This protein, in association with others, is essential for lymphocyte formation. Thus, the mutation of the IL2Rγ gene causing a blockage in production of the IL2-Rγ receptor will thereby also block cell differentiation into lymphocytes, mainly T and Natural Killer (NK) lymphocytes. Similarly, the absence of T and NK lymphocytes in the body leads to inactivation of the B lymphocytes formed.

The IL2Rγ gene is located on the X chromosome. Males with only one copy of this chromosome, and therefore of the IL2Rγ gene, are automatically affected by the disease if the gene is mutated. On the other hand, females with 2 copies of the gene are said to be “healthy carriers” and will only show symptoms of the disease in extremely rare cases.

Symptoms of SCID-X generally appear between 3 and 6 months of age. Most affected children suffer from stunted growth, oral and genital rashes, and multiple persistent infections leading to death within 1 or 2 years if left untreated.

The prevalence of SCID-X is very difficult to assess due to a lack of diagnosis, but has been estimated at around 1 in 50,000 births.

The gold standard treatment is immune reconstitution via bone marrow transplantation. The success rate of this approach is directly linked to the level of compatibility between the marrow donor and the sick child. If compatibility is high (usually marrow from the patient's brother or sister), total remission will be achieved in 20% of cases. However, in 80% of cases, the patient will develop variably intense symptoms of GVHD (Graft Versus Host Disease), meaning that the immune cells of the transplanted marrow will attack the patient.

The alternative treatment is gene therapy based on the use of viruses (lentivirus or AAV), where the IL2Rγ gene is reintroduced into a sample taken from the patient's hematopoietic stem cells and then reinjected to repopulate the immune system. This approach, still in clinical trials, claims to be effective in 80% of cases, but requires several sessions of extremely costly treatment, and in 20% of cases leads to the development of leukemia due to the fact that this viral approach is not targeted and does not bring the new IL2Rγ gene to its original locus.

Patent application US20150152436A1 describes new therapeutic developments aimed at adapting the CRISPR/CAS9 genome-editing approach to the treatment of SCID-X. However, the polymorphism associated with this pathology, as well as the possible insertion of the IL2Rγ cDNA by more than 600 base pairs, remains a significant technical obstacle to clinical use.

The need to provide effective treatment therefore remains.

One of the aims of the invention is to overcome the drawbacks of the prior art.

One aim of the invention is to propose a composition capable of enabling the replacement of the IL2Rγ gene in an efficient and complete manner.

A further aim of the invention is to provide a method or drug capable of treating pathologies linked to the deficiency of said gene.

### SUMMARY

The invention relates to a composition comprising:
a first single-stranded nucleic acid molecule comprising or consisting essentially of an A sequence allowing the insertion of a complementary sequence of a nucleic acid of interest, or comprising a complementary sequence of a nucleic acid of interest, said complementary sequence binding at 5′ to a first T-rich sequence of 40 to 60 nucleotides in length and at 3′ to a second T-rich sequence of 40 to 60 nucleotides in length, said first and second T-rich sequences respectively comprising a first and a second domain of 6 to 12 G/C-rich nucleotides, the sequence of the first domain being complementary to the sequence of the second domain, said first and second domains being positioned 15 to 52 nucleotides from said A sequence, said first molecule comprising at its 5′ end at least one first sequence oriented 5′-to-3′ for recognizing a transposase and at its 3′ end a second sequence for recognizing said transposase,
a second single-stranded nucleic acid molecule comprising or consisting essentially of a B sequence allowing the insertion of a complementary sequence of a nucleic acid of interest, or comprising a complementary sequence of a nucleic acid of interest, said complementary B sequence binding at 5′ to a third T-rich sequence of 40 to 60 nucleotides in length and at 3′ to a fourth T-rich sequence of 40 to 60 nucleotides in length, said third and fourth T-rich sequences respectively comprising a third and a fourth domain of 6 to 12 G/C-rich nucleotides, the sequence of the third domain being complementary to the sequence of the fourth domain, said third and fourth domains being positioned 15 to 52 nucleotides from said B sequence, said second molecule comprising at its 5′ end at least one first sequence oriented 5′-to-3′ for recognizing said transposase and at its 3′ end the second sequence for recognizing said transposase,

said B sequence being a complementary sequence of said nucleic acid of interest, the A sequence being positioned at 5′ of a region of interest of said nucleic acid of interest and the B sequence positioned at 3′ of the region of interest of said nucleic acid of interest, and
a third single-stranded molecule comprising
in its 5′ part, at least one complementary sequence of said second sequence for recognizing said transposase of the first molecule,
in its 3′ part, at least one complementary sequence of said first sequence for recognizing said transposase of the second molecule, and
an intermediate region located between the complementary sequence of said second recognition sequence of said transposase of the first molecule and the complementary sequence of said first recognition sequence of said transposase of the second molecule, said intermediate region comprising a sequence coding for a portion of the interleukin-2 receptor gamma or IL2-Rγ, or coding for the complete IL2-Rγ receptor,

the first and third single-stranded nucleic acid molecules being paired according to the base complementarity defined by Watson and Crick so as to define two double-stranded binding sites of said transposase, and the second and third single-stranded nucleic acid molecules being paired according to the base complementarity defined by Watson and Crick so as to define two double-stranded binding sites of said transposase.

The invention is based on the inventors' observation that the use of a complex molecule as described above, involving a bacterial transposase, enables targeted and effective replacement of the IL2Rγ gene, which codes for IL2-Rγ.

This means that the invention relates to a molecular complex comprising a first single-stranded nucleic acid molecule, a second single-stranded nucleic acid molecule, and a third single-stranded nucleic acid molecule, said third single-stranded nucleic acid molecule comprising or consisting essentially at its 5′ end of at least one complementary sequence of said first recognition sequence of said transposase, and said third single-stranded nucleic acid molecule comprising or consisting essentially at its 3′ end of at least one sequence complementary to said second recognition sequence of said transposase,
said complex being such that the first, second and third single-stranded nucleic acid molecules are paired according to the base complementarity defined by Watson and Crick so as to define two double-stranded binding sites for said transposase in the 5′ end of the third molecule and two double-stranded binding sites for said transposase in the 3′ end of the third molecule.

The invention is based on the unexpected observation made by the inventor that the use of specific single-stranded guides capable of targeting a region of a nucleic acid of interest makes it possible to mobilize transposases in a controlled and “site-specific” manner, and thus to use the recombination properties of said transposases to replace sequences in molecules of interest.

The aforementioned molecular complex is in fact the basic unit of the technology defined in the invention. This basic unit is useful for guiding the recombinases to a specific site where the recombination, and therefore the sequence replacement, must take place. Unlike the CRISPR/Cas9 system, which requires the presence of PAM-type (NGG) sequences, the molecular tool defined herein may be used on any target sequence, regardless of its sequence.

The aforementioned molecular complex is therefore the basic unit to be completed by:
a homology region of a 5′ region of the target sequence, said homology region being represented by A sequence and
a homology region of a 3′ region of the target sequence, said homology region being represented by B sequence.

This is therefore an intermediate product of the tool as described hereinafter.

The molecular complex consists of three single-stranded nucleic acid molecules, which may be DNA molecules, RNA molecules or mixed RNA and DNA molecules.

These three molecules are partially complementary with one another in pairs, according to the base complementarity of nucleic acids defined by Watson and Crick, that is to say that adenine pairs with thymidine or uracil, and cytosine pairs with guanine, and vice versa.

More particularly, the three molecules forming the aforementioned complex each comprise the sequence of one of the strands of a double-stranded molecule corresponding to the binding sequence of a transposase. Also, each single-stranded molecule therefore comprises a transposase binding “half sequence” and therefore cannot allow an interaction with said corresponding transposase. On the other hand, when the three molecules of the complex interact together, by base pairing as defined hereinbefore, a double-stranded molecule is thus formed, reconstituting a double-stranded binding site of said transposase, the latter thus being able to interact with the molecule formed.

### BRIEF DESCRIPTION OF THE FIGURES

The invention will be better understood from reading the following examples and figures:

FIG. 1A is a first schematic representation of a first form of pairing of the first, second and third molecules of the composition according to the invention.

FIG. 1B is a second schematic representation of a first form of pairing of the first, second and third molecules of the composition according to the invention.

FIG. 1C is a third schematic representation of a first form of pairing of the first, second and third molecules of the composition according to the invention.

FIG. 1D is a fourth schematic representation of a first form of pairing of the first, second and third molecules of the composition according to the invention.

FIG. 1E is a fifth schematic representation of a first form of pairing of the first, second and third molecules of the composition according to the invention.

FIGS. 2A-2C are diagrams showing the principle and steps involved in single-stranded replacement using the first, second and third molecules of the composition according to the invention.

FIG. 3A-3C are diagrams showing the principle and steps involved in double-stranded replacement using the first, second, third, fourth, fifth, and sixth molecules of the composition according to the invention.

FIG. 4 is a diagram showing the combination of molecules contained in tube 1 of example 1. The black ball represents biotin.

FIG. 5 is a diagram showing the combination of molecules contained in tube 2 of example 1. The black ball represents biotin.

### DETAILED DESCRIPTION

### The Complex

In addition to the detailed definition of each of the first, second and third molecules, each described below, the complex according to the invention is such that it comprises two pairs of binding sites to one or more transposases,
a first pair of binding sites to a first transposase as a result of pairing according to the base complementarity of nucleic acids defined by Watson and Crick between the first single-stranded nucleic acid molecule and the third nucleic acid molecule, and
a second pair of binding sites to a second transposase as a result of pairing according to the base complementarity of nucleic acids defined by Watson and Crick between the second single-stranded nucleic acid molecule and the third nucleic acid molecule.

the first and second transposases may be the same or different.

Each transposase-binding site consists of two double-stranded patterns, a first double-stranded pattern composed of a first transposase-binding half-site and a complementary sequence, according to the base complementarity defined by Watson and Crick, of said first transposase-binding half-site, and a second double-stranded pattern composed of a second transposase-binding half-site and a complementary sequence, according to the base complementarity defined by Watson and Crick, of said second transposase-binding half-site.

In the invention, the complementary sequence of a transposase-binding half-site is referred to as a complementary half-site of said site.

A transposase binding site therefore consists of four half-sites:
a first transposase-binding half-site with a first sequence,
a second transposase-binding half-site with a second sequence,
a third half-site with a sequence complementary to the sequence of the first half-site, according to the base complementarity defined by Watson and Crick, and
a fourth half-site with a sequence complementary to the sequence of the second half-site, according to the base complementarity defined by Watson and Crick.

Thus, in the complex there will be 8 binding sites, four of them constituting the first pair of binding sites by pairing the first and third nucleic acid molecules, and the other four constituting the second pair of binding sites by pairing the second and third nucleic acid molecules.

Note that for the first pair of transposase binding sites, the four half-binding sites are such that
the first half-site and the second half-site are contained in the first nucleic acid sequence,
the complementary site of the second half-site is comprised in the third nucleic acid sequence,

their positions in the complex are therefore defined, and
the complementary site of the first half-site is either i) comprised in the first nucleic acid molecule, ii) or comprised in the third nucleic acid molecule

it is therefore not necessary to define its position in one (first) or the other (third) nucleic acid molecule, the essential point being that when the first and third nucleic acid molecules are paired according to the base complementarity defined by Watson and Crick, the two half-sites are reconstituted, that is, the first half-site is paired with the half-site complementary to the first half-site, and the second half-site is paired with the half-site complementary to the second half-site.

Note also that for the second pair of transposase binding sites, the four half-binding sites are such that
the first half-site and the second half-site are contained in the second nucleic acid sequence,
the complementary site of the first half-site is comprised in the third nucleic acid sequence, their positions in the complex are therefore defined, and
the complementary site of the second half-site is either i) comprised in the second nucleic acid molecule, ii) or comprised in the third nucleic acid molecule

it is therefore not necessary to define its position in one (second) or the other (third) nucleic acid molecule, the essential point being that when the second and third nucleic acid molecules are paired according to the base complementarity defined by Watson and Crick, the two half-sites are reconstituted, that is, the first half-site is paired with the half-site complementary to the first half-site, and the second half-site is paired with the half-site complementary to the second half-site.

### The First Molecule.

The first molecule of the complex is the molecule that comprises, once modified, a nucleic acid sequence that makes it possible to specifically target a region of interest of a nucleic acid molecule of interest. This sequence of interest is selected by the user of the system according to the selected target. This sequence of interest is inserted into the first molecule of said complex at the A region. This A region corresponds at least to two nucleic acids between which the sequence that makes it possible to target the target molecule is inserted. In view of the oriented structure of the nucleic acids (5′-to-3′ direction), it is important for the sequence that makes it possible to target the region of interest to be positioned in the correct direction, in order to allow pairing with the target sequence.

Also, advantageously, the A region comprises one or more sites recognizing restriction enzymes in order to promote an oriented insertion. One or more of the following sites may be present in the A region:

TABLE 1

| | | | |
|---|---|---|---|
| | | AA/CGTT | AclI |
| | | A/AGCTT | HindIII |
| | | AAT/ATT | SspI |
| | | /AATT | MluCI |
| | | A/CATGT | PciI |
| | | A/CCGGT | AgeI |
| | | ACCTGC(4/8) | BfuAI |
| | | | BspMI |
| | | A/CCWGGT | SexAI |
| | | A/CGCGT | MluI |
| | | ACGGC(12/14) | BceAI |
| | | A/CGT | HpyCH4IV |
| | | ACN/GT | HpyCH4III |
| SEQ ID NO: | 4 | (10/15)ACNNNNGTAYC(12/7) | BaeI |
| SEQ ID NO: | 5 | (9/12)ACNNNNNCTCC(10/7) | BsaXI |
| | | A/CRYGT | AflIII |
| | | A/CTAGT | SpeI |
| | | ACTGG(1/−1) | BsrI |
| | | ACTGGG(5/4) | BmrI |
| | | A/GATCT | BglII |
| | | AGC/GCT | AfeI |
| | | AG/CT | AluI |
| | | AGG/CCT | StuI |
| | | AGT/ACT | ScaI- |
| | | AT/CGAT | ClaI BspDI |
| SEQ ID NO: | 6 | ATCTATGTCGGGTGCGGAGAAAGAGG | PI-SceI |
| | | TAAT(−15/−19) | |
| | | ATGCA/T | NsiI |
| | | AT/TAAT | AseI |
| | | ATTT/AAAT | SwaI |
| SEQ ID NO: | 7 | (11/13)CAANNNNNGTGG(12/10) | CspCI |
| | | C/AATTG | MfeI |
| | | CACCTGC(4/8) | PaqCI |
| | | CACGAG | Nb.BssSI |
| | | CACGAG(−5/−1) | BssSI-v2 |
| | | CACGTC(−3/−3) | BmgBI |
| | | CAC/GTG | PmlI |
| | | CACNNN/GTG | DralII |
| SEQ ID NO: | 8 | CACNN/NNGTG | AleI-v2 |
| | | CAGCAG(25/27) | EcoP151 |
| | | CAG/CTG | PvuII |
| | | CAGNNN/CTG | AlwNI |
| | | CAGTG(2/0) | BtsIMutI |
| | | CA/TATG | NdeI |
| | | CATG/ | NlalII |
| | | /CATG | FatI |
| | | C/ATG | CviAIII |
| SEQ ID NO: | 9 | CAYNN/NNRTG | MslI |
| | | CC(12/16) | FspEI |
| SEQ ID NO: | 10 | CCANNNNN/NNNNTGG | XcmI |
| SEQ ID NO: | 11 | CCANNNNN/NTGG | BstXI |
| SEQ ID NO: | 12 | CCANNNN/NTGG | PfIMI |
| | | CCATC(4/5) | BccI |
| | | C/CATGG | NcoI |
| | | CCCAGC(−5/−1) | BseYI |
| | | CCCGC(4/6) | FauI |
| | | CCC/GGG | SmaI |
| | | C/CCGGG(0/−1)CCD | TspMI XmaI |
| | | | Nt.CviPII |
| | | CCDG(10/14) | LpnPI |
| | | CCGC(−3/−1) | AciI |
| | | CCGC/GG | SaclI |
| | | CCGCTC(−3/−3) | BsrBI |
| | | C/CGG | MspI HpalI |
| | | CC/NGG | ScrFI |
| | | /CCNGG | StyD4I |
| | | C/CNNGG | BsaJI |
| SEQ ID NO: | 13 | CCNNNNN/NNGG | BslI |
| | | C/CRYGG | BtgI |
| | | CC/SGG | NciI |
| | | C/CTAGG | AvriI |
| | | CCTC(7/6) | MnlI |
| | | CCTCAGC | Nb.BbvCI |
| | | CCTCAGC(−5/−7) | Nt. BbvCI |
| | | CCTCAGC(−5/−2) | BbvCI |
| | | CCTGCA/GG | SbfI |
| | | CCTNAGC(−5/−2) | Bpu10I |
| | | CC/TNAGG | Bsu36I |
| SEQ ID NO: | 14 | CCTNN/NNNAGG | EcoNI |
| | | CCTTC(6/5) | HpyAV |
| | | /CCWGG | PspGI |
| | | CC/WGG | BstNI |
| | | C/CWWGG | StyI |
| SEQ ID NO: | 15 | (10/12)CGANNNNNNTGC(12/10) | BcgI |
| | | CGAT/CG | PvuI |
| | | CG/CG | BstUI |
| | | C/GGCCG | EagI |
| | | CG/GWCCG | RsrlI |
| | | CGRY/CG | BsiEI |
| | | C/GTACG | BsiWI |
| | | CGTCTC | BsmBI-v2 |
| | | CGTCTC(1/5) | Esp3I |
| | | CGWCG/ | Hpy99I |
| | | CMG/CKG | MspA1I |
| SEQ ID NO: | 16 | CNNNNNNNNNNN/NNNNNNNNNG | AbaSI |
| | | CNNR(9/13) | MspJI |
| | | CR/CCGGYG | SgrI |
| | | C/TAG | BfaI |
| | | CTCAG(9/7) | BspCNI |
| | | C/TCGAG | XhoI PaeR7I |
| | | CTCTTC(1/4) | EarI |
| | | CTGAAG(16/14) | AcuI |
| | | CTGCA/G | PstI |
| | | CTGGAG(16/14) | BpmI |
| | | C/TNAG | DdeI |
| | | C/TRYAG | SfcI |
| | | C/TTAAG | AflII |
| | | CTTGAG(16/14) | BpuEI |
| | | C/TYRAG | SmlI |
| | | C/YCGRG | BsoBI AvaI |
| | | GAAGA(8/7) | MbolI |
| | | GAAGAC(2/6) | BbsI |
| SEQ ID NO: | 17 | GAANN/NNTTC | XmnI |
| | | GAATGC(1/−1) | BsmI |
| | | GAATGC | Nb.BsmI |
| | | G/AATTC | EcoRI |
| | | GACGC(5/10) | HgaI |
| | | GACGT/C | AatlI |
| | | GAC/GTC | ZraI |
| | | GACN/NNGTC | PfIFI Tth111I |
| SEQ ID NO: | 18 | GACNN/NNGTC | PshAI |
| SEQ ID NO: | 19 | GACNNN/NNGTC | AhdI |
| SEQ ID NO: | 20 | GACNNNN/NNGTC | DrdI |
| | | GAG/CTC | Eco53kI |
| | | GAGCT/C | SacI |
| | | GAGGAG(10/8) | BseRI |
| | | GAGTC(4/-5) | Nt. BstNBI |
| | | GAGTC(4/5) | PleI |
| | | GAGTC(5/5) | MlyI |
| | | G/ANTC | HinfI |
| | | GAT/ATC | EcoRV |
| | | GA/TC | DpnI |
| | | /GATC | Sau3A−DpnII |
| | | | MboI |
| SEQ ID NO: | 21 | GATNN/NNATC | BsaBI |
| | | G/AWTC | TfiI |
| | | GCAATG | Nb.BsrDI |
| | | GCAATG(2/0) | BsrDI |
| | | GCAGC(8/12) | BbvI |
| | | GCAGTG(2/0) | BtsI-v2 |
| | | GCAGTG | Nb.BtsI |
| SEQ ID NO: | 22 | GCANNNN/NTGC | BstAPI |
| | | GCATC(5/9) | SfaNI |
| | | GCATG/C | SphI |
| | | GCCC/GGGC | SrfI |
| | | GCCGAG(21/19) | NmeAlII |
| | | G/CCGGC | NgoMIV |
| | | GCC/GGC | NaeI |
| SEQ ID NO: | 23 | GCCNNNN/NGGC | BglI |
| | | GCGAT/CGC AsiSI | |
| | | GCGATG(10/14) | BtgZI |
| | | GCG/C | HhaI |
| | | G/CGC | HinP1I |
| | | G/CGCGC | BssHII |
| | | GC/GGCCGC | NotI |
| | | GC/NGC | Fnu4HI |
| | | GCN/NGC | Cac8I |
| SEQ ID NO: | 24 | GCNNNNN/NNGC | MwoI |
| | | G/CTAGC | NheI |
| | | GCTAG/C | BmtI |
| | | GCTCTTC(1/-7) | Nt. BspQI |
| | | GCTCTTC(1/4) | Sapl BspQI |
| | | GC/TNAGC | BlpI |
| | | G/CWGC | ApeKI TseI |
| | | GDGCH/C | Bsp1286I |
| | | GGATC(4/5) | AlwI |
| | | GGATC(4/−5) | Nt.AlwI |
| | | G/GATCC | BamHI |
| | | GGATG(9/13) | FokI |
| | | GGATG(2/0) | BtsCI |
| | | GG/CC | HaelII |
| | | GGCCGG/CC | FseI |
| SEQ ID NO: | 25 | GGCCNNNN/NGGCC | SfiI |
| | | G/GCGCC | KasI |
| | | GG/CGCC | NarI |
| | | GGCGC/C | PluTI |
| | | GGC/GCC | SfoI |
| | | GG/CGCGCC | AscI |
| | | GGCGGA(11/9) | EciI |
| | | GGGAC(10/14) | BsmFI |
| | | GGGCC/C | ApaI |
| | | G/GGCCC | PspOMI |
| | | G/GNCC | Sau96I |
| | | GGN/NCC | NlaIV |
| | | G/GTACC | Acc65I |
| | | GGTAC/C | KpnI |
| | | GGTCTC(1/5) | BsaI v2 |
| | | GGTGA(8/7) | HphI |
| | | G/GTNACC | BstEII |
| | | G/GWCC | AvalI |
| | | G/GYRCC | BanI |
| | | GKGCM/C | BaeGI |
| | | GR/CGYC | BsaHI |
| | | GRGCY/C | BanlI |
| | | GT/AC | RsaI |
| | | G/TAC | CviQI |
| | | GTATAC | BstZ17I |
| | | GTATCC(6/5) | BciVI |
| | | G/TCGAC | SalI |
| | | GTCTC(1/5) | BsmAI Bco |
| | | | DI |
| | | GTCTC(1/−5) | Nt. BsmAI |
| | | G/TGCAC | ApaLI |
| | | GTGCAG(16/14) | BsgI |
| | | GT/MKAC | AccI |
| | | GTN/NAC | Hpy166II |
| | | /GTSAC | Tsp45I |
| | | GTT/AAC | HpaI |
| | | GTTT/AAAC | PmeI |
| | | GTY/RAC | HincII |
| | | GWGCW/C | BsIHKAI |
| | | NNCASTGNN/ | TspRI |
| | | R/AATTY | ApoI |
| | | RCATG/Y | NspI |
| | | R/CCGGY | BsrFI-v2 |
| | | R/GATCY | BstYI |
| | | RGCGC/Y | HaelI |
| | | RG/CY | CviKI-1 |
| | | RG/GNCCY | EcoO109I |
| | | RG/GWCCY | PpuMI |
| SEQ ID NO: | 26 | TAACTATAACGGTCCTAAGGTAGCGAA | I-CeuI |
| | | (−9/−13) | |
| | | TAC/GTA | SnaBI |
| SEQ ID NO: | 27 | TAGGGATAACAGGGTAAT(−9/−13) | I-SceI |
| | | T/CATGA | BspHI |
| | | T/CCGGA | BspEI |
| | | TCCRAC(20/18) | MmeI |
| | | T/CGA | TaqI-v2 |
| | | TCG/CGA | NruI |
| | | TCN/GA | Hpy188I |
| | | TC/NNGA | Hpy188III |
| | | T/CTAGA | XbaI |
| | | T/GATCA | BelI |
| | | TG/CA | HpyCH4V |
| | | TGC/GCA | FspI |
| SEQ ID NO: | 28 | TGGCAAACAGCTATTATGGGTATTATG | PI-PspI |
| | | GGT(−13/−17) | |
| | | TGG/CCA | MscI |
| | | T/GTACA | BsrGI |
| | | T/TAA | MseI |
| | | TTAAT/TAA | PacI |
| | | TTA/TAA | PsiI-v2 |
| | | TT/CGAA | BstBI |
| | | TTT/AAA | DraI |
| | | VC/TCGAGB | PspXI |
| | | W/CCGGW | BsaWI |
| | | YAC/GTR | BsaAI |
| | | Y/GGCCR | EaeI |

Obviously, in the context of a chemical synthesis of the first molecule, it is not necessary to have cloning (or insertion) sites of the sequence making it possible to target the target region, but rather to take good care to provide a correctly oriented sequence. This is of course however possible.

The first molecule further consists, on either side of the A region, of A/T-rich sequences, or in the case of RNA, of A/U-rich sequences, in order to allow a certain flexibility of the structure. A/T-rich or A/U rich are understood in the invention to mean a sequence that comprises more than 50% of A or T, or U, preferably more than 50% of T or U, with respect to the total number of nucleotides that constitute the sequence. These sequences on either side of the A region have a size in nucleotides ranging from 10 nucleotides to 60 nucleotides.

The flexibility of these sequences flanking the A region, due to the presence of numerous A, T or U bases, may have the effect of allowing a recombination via the recombinases that is not sufficiently controlled, maybe even when the complex still has not recognized the target molecule.

Also, in order to overcome this problem, GC-rich sequences are introduced into each of the A/T-rich, especially T-rich, or A/U-rich, sequences bordering the A region. These G/C-rich regions consist of 6 to 12 nucleotides, in which the amount of C or G or G+C bases is greater than 50% of the nucleotides contained in said G/C-rich sequence.

In order to stabilize the structure of the first molecule and, as described hereinbefore, prevent inadvertent recombination, the G/C-rich regions are positioned 15 to 52 nucleotides from the end of the A region.

For greater clarity, if the A region consists of three nucleotides, the central nucleotide corresponding to position 0, the A/T-rich or A/U-rich region begins on the left at position-2, and on the right at position +2. Therefore, on the left, the G/C-rich region is positioned from position-17 to position-54 and, on the right, from position +17 to position +54.

Another important element: the G/C-rich sequence to the right (or 5′) of the A region is necessarily complementary (according to the Watson and Crick pairing rule) to the G/C-rich region to the right (or 3′) of the A region. Also, the first single-stranded molecule pairs with itself at the G/C-rich regions, which prevents any recombination by the transposases, as long as there is no interaction with the complementary target sequence of the region that is inserted into the A region of the first molecule.

Finally, the first molecule comprises at its 5′ end a sequence corresponding to a first site for binding to a transposase, and at its 3′ end a second site for binding to said transposase.

The first binding site and the second binding site are advantageously the same, and above all both correspond to the same strand of the double-stranded binding site of said transposase. This means that the first transposase binding site present in the 5′ region of the first molecule can only be paired integrally, and therefore stably, with the transposase binding site present in the 3′ region.

The first binding site and the second binding site are advantageously the same, but each correspond to a different strand of the double-stranded transposase binding site. Also, for example, if the first transposase binding site corresponds to the sense strand, the second transposase binding site corresponds to the sequence of the complementary strand. It is then possible to have two configurations: either i) the second binding site which corresponds to the complementary strand is oriented in the 3′-to-5′ direction, in which case it is able to pair with the first transposase binding site and form the double-stranded site, or ii) the second binding site which corresponds to the complementary strand is oriented in the 5′-to-3′ direction, in which case it is not able to pair with the first transposase binding sequence, due to their orientation not being complementary. In the aforementioned case i), if the first single-stranded molecule pairs with itself at the first and second binding sites, it is not possible to form the aforementioned complex, since there are no more single-stranded complementary regions available to pair with the second molecule so as to form two double-stranded transposase binding sites.

Also, the first molecule, when it lacks a complementary sequence of the target region in the A part, or when it contains such a target sequence but the latter does not interact (does not pair) with said target sequence, forms a three-dimensional structure wherein the entire molecule is single-stranded with the exception of the region corresponding to the G/C-rich regions that pair with one another.

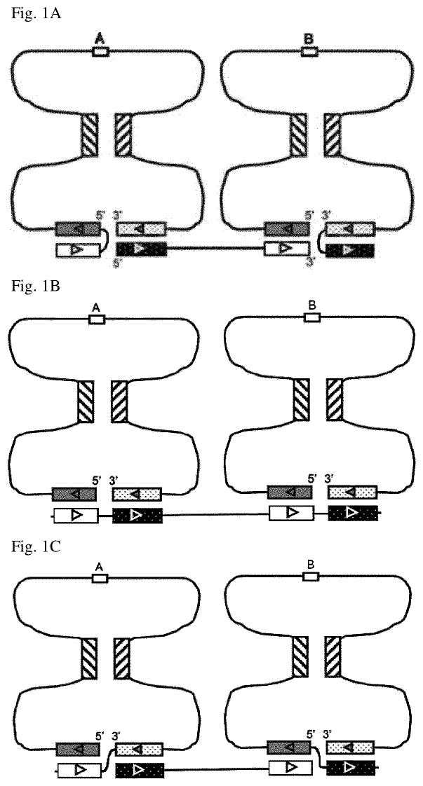

A schematic representation in paired form is shown in FIGS. 1A to 1E.

### The Second Molecule.

The second molecule in the complex is structurally similar to the first, so the above explanations apply mutatis mutandis. However, in the second molecule, the B region (or B sequence) and the transposase binding sites are organized differently.

First of all, the B region is different from the A region of the first molecule. Indeed, for the purposes of directed recombination, there can be no competition for the same target of interest between the first and second molecules.

It is therefore necessary for the B region to correspond to a second sequence complementary to the target sequence, this second sequence complementary to the target sequence being positioned at 3′ relative to the first sequence recognized by the complementary sequence corresponding to A sequence of the first molecule.

Consequently, when the first molecule and the second molecule are paired with the target sequence, the target sequence will be bordered by these two molecules, with the first molecule located at 5′ and the second molecule positioned at 3′. The region of the target sequence located between the region of interaction with the first molecule and the region of interaction with the second molecule corresponds to the sequence that will be replaced by that of the third molecule.

### The Third Molecule

The third molecule in the above-mentioned complex is simpler than the first two (the first and second molecules). The third molecule comprises, in its 5′ part, a transposase binding site which is complementary to the site for binding to said transposase present in the 3′ part of the first molecule. Therefore, when the complex is formed, the (single-stranded) transposase binding half-site located at 3′ of the first molecule may pair with the (single-stranded) transposase binding half-site located at 5′ of the second molecule so as to form a double-stranded transposase binding site, a double-stranded site on which the transposase can bind.

The 3′ part of the third molecule contains a transposase binding site which is complementary to the site for binding to said transposase present in the 5′ part of the second molecule. Therefore, when the complex is formed, the (single-stranded) transposase binding half-site located at 3′ of the third molecule may pair with the (single-stranded) transposase binding half-site located at 5′ of the second molecule so as to form a double-stranded transposase binding site, a double-stranded site on which the transposase can bind.

Between the 5′ part, which comprises a transposase-binding half-site, and the 3′ part, which comprises a transposase-binding half-site, the third molecule comprises a replacement sequence, that is, the sequence that will eventually replace the target sequence. This replacement sequence is bordered at 5′ by at least one restriction site and at 3′ by at least one restriction site; these two restriction sites being distinct. Also, the third molecule comprises in the 5′ to 3′ direction: a transposase binding half-site complementary to the transposase binding site of the first molecule, followed by at least one restriction site, followed by the replacement sequence, followed by at least one restriction site different from the restriction site upstream of the replacement sequence, followed finally by a transposase binding half-site complementary to the transposase binding site of the second molecule.

The replacement sequence corresponds to all or part of the gene coding for the IL2-Rγ protein, that is, all or part of the ILR2γ gene.

The common gamma chain (γc) (or CD132), is also known as the interleukin-2 receptor gamma subunit or IL-2RG, or IL2-Rγ. It is a cytokine receptor subunit that is common to the receptor complexes of at least six different interleukin receptors: interleukin 2 (IL-2), interleukin 4 (IL-4), interleukin 7 (IL-7), interleukin 9 (IL-9), interleukin 15 (IL-15) and the interleukin-21 receptor. This chain is a glycoprotein belonging to the type I cytokine receptor family expressed on most lymphocyte populations. The ILR2γ gene is located on the mammalian X chromosome.

IL2-Rγ is expressed on the surface of immature blood cells in bone marrow. One end of the protein resides outside the cell where it binds cytokines, and the other end of the protein resides inside the cell where it transmits signals to the cell nucleus. The common gamma chain associates with other proteins to direct hematopoietic cells to form lymphocytes. The receptor also directs the growth and maturation of lymphocyte subtypes: T lymphocytes, B lymphocytes and natural killer (NK) cells.

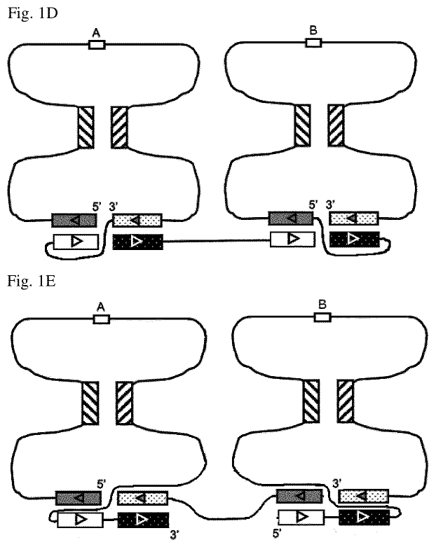

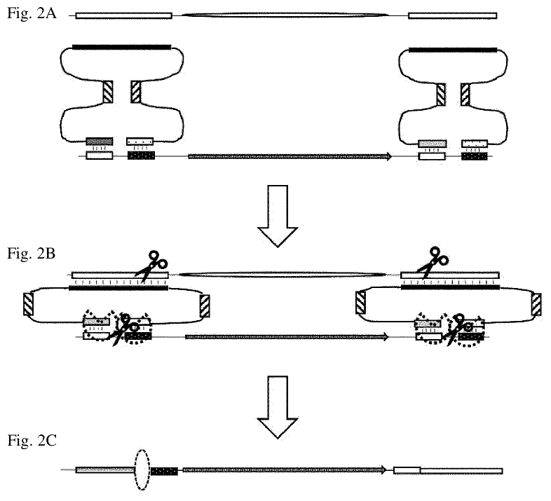

The composition according to the invention makes it possible to obtain the above-mentioned complex. [FIG. 2A-2C] schematically show the complex formed between the first, second and third molecules according to the invention.

The complex formed from molecules of the composition of the invention, when the three molecules are correctly paired, comprises two pairs of transposase recognition double-stranded binding sites:
the first pair being obtained by the hybridization of the first molecule with the third molecule, and
the second pair being obtained by the hybridization of the second molecule with the third molecule.

The A sequence contained in the first molecule is complementary to the same strand of the nucleic acid which is complementary to the B sequence contained in the second molecule. In other words, the A sequence contained in the first molecule and the B sequence contained in the second molecule are capable of hybridizing with the same nucleic acid simultaneously, since the two sequences A and B do not recognize the same sequence.

In order to further clarify the remarks, the interest in the present invention is to propose a first molecule and a second molecule, both as defined hereinbefore, the respective sequences A and B being such that they are capable of recognizing, for one, a sequence located at 5′ of the target sequence of the nucleic acid of interest and, for the other, a sequence located at 3′ of the same target sequence of the nucleic acid of interest. The sequences A and B are therefore complementary to regions bordering the sequence of interest, which it is sought to replace, of the nucleic acid molecule of interest.

The first and second molecules of interest are therefore essential for targeting specifically the molecule of interest, in order to flank the sequence to be replaced.

The third molecule in the ensemble, in turn, is the one that provides the nucleic acid molecule containing the replacement sequence, that is, a sequence corresponding to all or part of the gene coding for IL2-Rγ.

From a mechanistic point of view, the complex according to the invention is such that it consists of its three molecules, the first and second molecules being structurally organized in space so that their G/C-rich regions are paired.

On either side of the third molecule, that is to say at 5′ and at 3′, due to the hybridization with the first and second molecules, two pairs of sites for binding to a transposase allow, when transposase is present, transposase dimers to bind to the ensemble.

It will be noted that the transposase binding sites of the first molecule can be the same as those of the second molecule, or different. In the case where the binding sequences are the same, the transposases at 5′ of the third molecule (by hybridization of the 5′ part of the third molecule with the first molecule), and at 3′ of the third molecule (by hybridization of the 3′ part of the third molecule with the second molecule) are the same. Also, by way of example, if the binding sites are all binding sites for the Tn5 transposase, the ensemble is associated with two Tn5 transposase dimers.

It is also possible for the binding sites of the first molecule and of the second molecule not to recognize the same transposase. In this case, and according to the definition given hereinbefore, the 5′ part of the third molecule forms, by hybridization with the first molecule, two double-stranded sites for binding to a first transposase, and the 3′ part of the third molecule forms, by hybridization with the second molecule, two double-stranded sites for binding to a second transposase.

If the aforementioned complex, linked to two transposase dimers, is next brought into the presence of a nucleic acid molecule of interest, the 5′ part of which is complementary to the A sequence of the first molecule of the ensemble and the 3′ part of which is complementary to the B sequence of the second molecule of the ensemble, then the nucleic acid molecule of interest pairs with the ensemble at the above-described regions A and B. This interaction has the consequence of breaking the interaction of the two G/C-rich sequences of each of the first and second molecules of the ensemble. Therefore, the third molecule and the nucleic acid molecule of interest are moved closer together in space and the transposases can exercise their tagmentation activity, the result of which is the replacement of the sequence of the molecule of interest, bordered by the complementary sequences of the sequences A and B, by the sequence of the third molecule of the ensemble, which is located between the half-sites for binding to the transposase(s) at 5′ and at 3′.

In the invention, the first, second, and third molecule of the ensemble are advantageously molecules consisting of deoxyribonucleotides, in order to form single-stranded DNA molecules.

Even more advantageously, the first, second, and third molecules and third molecule of the ensemble are hybrid DNA/RNA molecules, wherein the “backbone” of the molecules is DNA, and the recognition sequences A and B of the nucleic acid molecule of interest and the central region of the third molecule are RNA. This is particularly advantageous when the sequence replacement that enables the invention is to be carried out directly on an RNA molecule.

FIG. 2A shows the interaction between the molecule of interest and the ensemble according to the invention.

Advantageously, the invention relates to the aforementioned complex, wherein said first molecule or said second molecule, or both molecules, are coupled with an enzyme, especially via a modified nucleotide. This enzyme is intended to promote the replacement of the molecule of interest. It may be:
a helicase, an enzyme capable of opening a double-stranded molecule that is supercoiled or even associated with proteins such as histones,
a topoisomerase, an enzyme acting on the topological structure of the DNA by generating transient breaks,
a ligase that forms a phosphodiester bond between the 5′ phosphate end of a nucleotide and the 3′ OH end of another nucleotide,
a polymerase, which synthesizes a nucleic acid molecule from an initiation site, free 3′OH, in the 5′->3′ direction, notably by copying an antiparallel complementary strand according to Watson and Crick's model.

It is also possible to combine two or more of said enzymes in order to have all the enzymatic material necessary to enable the sequence replacement contemplated in the scope of the invention.

In one particular aspect, the first molecule of the ensemble can contain in its 5′ part, more precisely between the first transposase binding site and the A region, one or more modified nucleotides. In the same way, the third molecule of the ensemble can contain in its 3′ part, more precisely between the first transposase binding site and the 3 region, one or more modified nucleotides.

This modified nucleotide is especially modified by grafting a substituted carbon chain with a protein tag, or else a molecule enabling a specific interaction such as streptavidin or biotin.

Such modifications then make it possible to specifically bind, to the first molecule of the ensemble, enzymes which can be useful for promoting tagmentation, and sequence replacement. It is particularly advantageous to have, for example, streptavidin grafting, which makes it possible to graft a biotinylated helicase (or conversely a helicase grafted to the streptavidin and a biotinylated nucleotide) useful for opening a double-stranded molecule. It is also possible to contemplate grafting with a biotinylated ligase (or coupled with streptavidin), in order to connect the recombinant strand on the 3′ side.

It is also possible to associate with the first molecule or with the second molecule of the ensemble an oligonucleotide, so that said oligonucleotide pairs on a predetermined region of said first or second molecule. This oligonucleotide is then advantageously coupled with a grafting molecule as explained hereinbefore.

Advantageously, the invention relates to the above-mentioned composition, wherein said A sequence comprises a sequence complementary to the sequence of a first region of the gene coding for IL2-Rγ, and wherein said B sequence comprises a sequence complementary to the sequence of a second region of the gene coding for IL2-Rγ, said A sequence and said B sequence being two different sequences, said first and second regions of the gene coding for IL2-Rγ, flanking a region comprising a sequence coding for a portion of IL2-Rγ, or coding for the complete IL2-Rγ receptor.

As explained above, in order to replace the target gene with the third molecule, it is advantageous for the sequences of part A of the first molecule and part B of the second molecule to be able:
to recognize the same target molecule, and
to frame the target region to be replaced, meaning that the first molecule must, via its A region, recognize a sequence upstream of the sequence to be replaced in the target molecule, and the second molecule must, via its B region, recognize a sequence downstream of the sequence to be replaced in the target molecule, or vice versa, that is, the first molecule must, via its A region, recognize a sequence downstream of the sequence to be replaced in the target molecule, and the second molecule must, via its B region, recognize a sequence downstream of the sequence to be replaced in the target molecule.

It is thus possible to replace any target sequence with any replacement sequence, as long as the A and B sequences of the first and second molecules respectively recognize the target region and frame it.

In the invention, it is particularly advantageous for all or part of the gene coding for IL2-Rγ to be replaced by the sequence of the third molecule. This is particularly advantageous when the target sequence comprises one or more mutations relative to the wild-type reference sequence, for example, a substitution of one or more contiguous or spaced nucleotides, a deletion of one or more contiguous or spaced nucleotides, or an insertion of one or more contiguous or spaced nucleotides.

The entire gene coding for IL2-Rγ is taken to mean the entire sequence of said gene including the 5′, 3′ regulatory elements, exons and introns.

In the context of the invention, “parts of the gene coding for IL2-Rγ” is taken to mean a part of the gene which does not enable the IL2-Rγ protein to be fully encoded. This may include, but is not limited to, an intro, an exon, several introns and exons, without the entire translational unit being complete, etc. It may be particularly advantageous to replace only part of the gene coding for IL2-Rγ, for example to replace only one exon in which a mutation is present. In this case, it may be advantageous to choose a sequence complementary to the A region of the first molecule in the intron preceding the sequence to be replaced, and a complementary part of the B region in the intron following the exon to be replaced. Of course, depending on the desired substitution, the person skilled in the art will be able to choose between the whole gene or a part of the gene, and thus define the most relevant A and B regions.

Advantageously, the invention relates to the composition as defined hereinbefore, wherein said first molecule comprises at its 5′ end a first sequence oriented 5′-to-3′ for recognizing a transposase and at its 3′ end a second sequence oriented 5′-to-3′ for recognizing said transposase and
wherein the third molecule comprises at its 5′ end a first complementary sequence of said first sequence for recognizing said transposase of the first molecule followed by a second complementary sequence of said second sequence for recognizing said transposase of the first molecule.

Even more advantageously, the invention relates to the composition as defined hereinbefore, wherein said first molecule comprises at its 5′ end a first sequence oriented 5′-to-3′ for recognizing a transposase and at its 3′ end a second sequence oriented 5′-to-3′ for recognizing said transposase,
said second molecule comprises at its 5′ end a first sequence oriented 5′-to-3′ for recognizing a transposase and at its 3′ end a second sequence oriented 5′-to-3′ for recognizing said transposase, and
wherein the third molecule comprises at its 5′ end a first complementary sequence of said first sequence for recognizing said transposase of the first molecule followed by a second complementary sequence of said second sequence for recognizing said transposase of the first molecule, and at its 3′ end, a first sequence complementary to said first sequence for recognizing said transposase of the second molecule followed by a second sequence complementary to said second sequence for recognizing said transposase of the second molecule.

A possible format for the first and second molecules of the invention is defined here. This configuration is shown in [FIG. 1B]. Of course, in this example of the configuration of the first, second and third molecules, between the two transposase half-sites of the 5′ and 3′ parts of the third molecule is the replacement sequence, in this case all or part of the gene coding for IL2-Rγ.

Advantageously, the invention relates to the aforementioned composition, wherein said first molecule comprises at its 5′ end a first sequence oriented 5′-to-3′ for recognizing a transposase and at its 3′ end a second sequence for recognizing said transposase, followed by a first complementary sequence of said first sequence for recognizing said transposase of the first molecule and
wherein the third molecule comprises at its 5′ end a complementary sequence of said second sequence for recognizing said transposase of the first molecule.

Even more advantageously, the invention relates to the aforementioned composition, wherein said first molecule comprises at its 5′ end a first sequence oriented 5′-to-3′ for recognizing a transposase and at its 3′ end a second sequence for recognizing said transposase, followed by a first complementary sequence of said first sequence for recognizing said transposase of the first molecule,
the second molecule comprises at its 5′ end a first sequence oriented 5′-to-3′ for recognizing a transposase and at its 3′ end a second sequence for recognizing a transposase, said first sequence for recognizing said transposase of the 5′ end being preceded by a second sequence complementary to said second sequence for recognizing said transposase of the first molecule,
and
wherein the third molecule comprises at its 5′ end a complementary sequence of said second sequence for recognizing said transposase of the first molecule and at its 3′ end a complementary sequence of said first sequence for recognizing said transposase of the second molecule.

Another possible format for the first and second molecules of the invention is defined here. This configuration is shown in [FIG. 1C] and [FIG. 1D]. Of course, in this example of the configuration of the first, second and third molecules, between the two transposase half-sites of the 5′ and 3′ parts of the third molecule is the replacement sequence, in this case all or part of the gene coding for IL2-Rγ.

Advantageously, the invention relates to the aforementioned composition, wherein said first molecule comprises at its 5′ end a first sequence oriented 5′-to-3′ for recognizing a transposase and at its 3′ end a second sequence complementary to said first sequence for recognizing said transposase of the first molecule, followed by a second sequence for recognizing said transposase, and
wherein the third molecule comprises at its 5′ end a complementary sequence of said second sequence for recognizing said transposase of the first molecule.

Even more advantageously, the invention relates to the aforementioned composition, wherein said first molecule comprises at its 5′ end a first sequence oriented 5′-to-3′ for recognizing a transposase and at its 3′ end a first sequence complementary to said first sequence for recognizing said transposase of the first molecule, followed by a second sequence for recognizing said transposase,
the second molecule comprises at its 5′ end a first sequence oriented 5′-3′ for recognizing a transposase and at its 3′ end a second sequence for recognizing said transposase of the first molecule, said first sequence being followed by a second sequence complementary to said second sequence for recognizing said transposase of the first molecule, and
wherein the third molecule comprises at its 5′ end a complementary sequence of said second sequence for recognizing said transposase of the first molecule and at its 3′ end a complementary sequence of the first sequence for recognizing the transposase of the second molecule.

This configuration is diagrammed in [FIG. 1E]. Of course, in this example of the configuration of the first, second and third molecules, between the two transposase half-sites of the 5′ and 3′ parts of the third molecule is the replacement sequence, in this case all or part of the gene coding for IL2-Rγ.

Advantageously, the invention relates to the above-mentioned composition, wherein said transposase is a bacterial transposase, especially a transposase selected from Tn5, Tn9, Tn10 or Tc1/mariner.

Advantageously, the aforementioned transposase is a bacterial-type transposase selected from the transposase of transposon Tn5, the transposase of transposon Tn9, the transposase of transposon Tn10, Tn903, Tn602, or even the transposase of the transposon Tc1, or more generally of the mariner transposon superfamily.

Other examples of transposases that can be used in the context of the invention are: the Vibrio harveyi transposase (transposase characterized by Agilent and used in the product SureSelect QXT), the MutA transposase and a Mu transposase recognition site comprising the terminal sequences R1 and R2, the transposase of Staphylococcus aureus transposon Tn552, the transposase of transposon Tn7, the Tn/O and IS10 transposase, the transposase of transposon Tn3.

The Tn5 transposase is the best known. It is coded by the Tnp gene of transposon Tn5. The transposase initiates the transposition by forming a transposase dimer which binds to its target sequences. In the context of this complex, the transposase then catalyzes four phosphoryl transfer reactions (DNA cleavage, DNA hairpin formation, hairpin resolution and strand transfer to the target DNA), resulting in the integration of the transposon into its new DNA site: this is what is known as tagmentation.

The invention is based on this tagmentation principle. By using the tagmentation properties of the transposases, it is possible to insert one sequence into another in a targeted manner, by virtue of the aforementioned complex.

Also in the context of the invention, when reference is made to a transposase, reference is being made to one of the aforementioned transposases, namely the transposases of transposons Tn5, Tn9, Tn10 or Tc1/mariner (or transposases mutated to increase their transposition or tagmentation activity).

In the invention, when several transposases are used simultaneously, one binding to the complex formed by the first molecule and the third molecule, and the other binding to the complex formed by the second molecule and the third molecule, the pairs of transposases resulting from transposons Tn5 and Tn10 are preferred.

Advantageously, the first and the second transposase recognition sequence are sequences for recognizing the Tn5 transposase having one of the following sequences:

(SEQ ID NO: 29)
-CTGtCTCTTataCAcAtcT,
(SEQ ID NO: 30)
-CTGACTCTTataCACAagT,
and
(SEQ ID NO: 31)
-CTGtCTCTTgatCAgATCT

As a result, the corresponding complementary sequences are as follows:

(SEQ ID NO: 32)
-AgaTgTGtatAAGAGaCAG, complementary to the
sequence SEQ ID NO: 29,
(SEQ ID NO: 33)
-ActTGTGtatAAGAGTCAG, complementary to the
sequence SEQ ID NO: 30,
and
(SEQ ID NO: 34)
-AGATCTGatcAAGAGaCAG, complementary to the
sequence SEQ ID NO: 31.

Other recognition sequences of a transposase are as follows:

Tn5MErev,
(SEQ ID NO: 35)
5′-[phos]CTGTCTCTTATACACATCT-3′
Tn5ME-A (Illumina FC-121-1030),
(SEQ ID NO: 36)
5′-TCGTCGGCAGCGTCAGATGTGTATAAGAGACAG-3′;
and
Tn5ME-B (Illumina FC-121-1031),
(SEQ ID NO: 37)
5′-GTCTCGTGGGCTCGGAGATGTGTATAAGAGACAG-3′

Other transposase recognition sequences are as follows:
sense sequence SEQ ID NO: i
antisense sequence SEQ ID NO: i+1,

wherein i ranges from 38 to 192.

This means for example that the following respective sense and antisense sequence pairs are considered: SEQ ID NO: 38 and SEQ ID NO: 39; SEQ ID NO: 40 and SEQ ID NO: 41, SEQ ID NO: 42 and SEQ ID NO: 43, SEQ ID NO: 44 and SEQ ID NO: 45, SEQ ID NO: 46 and SEQ ID NO: 47, SEQ ID NO: 48 and SEQ ID NO: 49, SEQ ID NO: 50 and SEQ ID NO: 51, SEQ ID NO: 52 and SEQ ID NO: 53, SEQ ID NO: 54 and SEQ ID NO: 55, SEQ ID NO: 56 and SEQ ID NO: 57, SEQ ID NO: 58 and SEQ ID NO: 59, SEQ ID NO: 60 and SEQ ID NO: 61, SEQ ID NO: 62 and SEQ ID NO: 63, SEQ ID NO: 64 and SEQ ID NO: 65, SEQ ID NO: 66 and SEQ ID NO: 67, SEQ ID NO: 68 and SEQ ID NO: 69, SEQ ID NO: 70 and SEQ ID NO: 71, SEQ ID NO: 72 and SEQ ID NO: 73, SEQ ID NO: 74 and SEQ ID NO: 75, SEQ ID NO: 76 and SEQ ID NO: 77, SEQ ID NO: 78 and SEQ ID NO: 79, SEQ ID NO: 80 and SEQ ID NO: 81, SEQ ID NO: 82 and SEQ ID NO: 83, SEQ ID NO: 84 and SEQ ID NO: 85, SEQ ID NO: 86 and SEQ ID NO: 87, SEQ ID NO: 88 and SEQ ID NO: 89, SEQ ID NO: 90 and SEQ ID NO: 91, SEQ ID NO: 92 and SEQ ID NO: 93, SEQ ID NO: 94 and SEQ ID NO: 95, SEQ ID NO: 96 and SEQ ID NO: 97, SEQ ID NO: 98 and SEQ ID NO: 99, SEQ ID NO: 100 and SEQ ID NO: 101, SEQ ID NO: 102 and SEQ ID NO: 103, SEQ ID NO: 104 and SEQ ID NO: 105, SEQ ID NO: 106 and SEQ ID NO: 107, SEQ ID NO: 108 and SEQ ID NO: 109, SEQ ID NO: 110 and SEQ ID NO: 111, SEQ ID NO: 112 and SEQ ID NO: 113, SEQ ID NO: 114 and SEQ ID NO: 115, SEQ ID NO: 116 and SEQ ID NO: 117, SEQ ID NO: 118 and SEQ ID NO: 119, SEQ ID NO: 120 and SEQ ID NO: 121, SEQ ID NO: 122 and SEQ ID NO: 123, SEQ ID NO: 124 and SEQ ID NO: 125, SEQ ID NO: 126 and SEQ ID NO: 127, SEQ ID NO: 128 and SEQ ID NO: 129, SEQ ID NO: 130 and SEQ ID NO: 131, SEQ ID NO: 132 and SEQ ID NO: 133, SEQ ID NO: 134 and SEQ ID NO: 135, SEQ ID NO: 136 and SEQ ID NO: 137, SEQ ID NO: 138 and SEQ ID NO: 139, SEQ ID NO: 140 and SEQ ID NO: 141, SEQ ID NO: 142 and SEQ ID NO: 143, SEQ ID NO: 144 and SEQ ID NO: 145, SEQ ID NO: 146 and SEQ ID NO: 147, SEQ ID NO: 148 and SEQ ID NO: 149, SEQ ID NO: 150 and SEQ ID NO: 151, SEQ ID NO: 152 and SEQ ID NO: 153, SEQ ID NO: 154 and SEQ ID NO: 155, SEQ ID NO: 156 and SEQ ID NO: 157, SEQ ID NO: 158 and SEQ ID NO: 159, SEQ ID NO: 160 and SEQ ID NO: 161, SEQ ID NO: 162 and SEQ ID NO: 163, SEQ ID NO: 164 and SEQ ID NO: 165, SEQ ID NO: 166 and SEQ ID NO: 167, SEQ ID NO: 168 and SEQ ID NO: 169, SEQ ID NO: 170 and SEQ ID NO: 171, SEQ ID NO: 172 and SEQ ID NO: 173, SEQ ID NO: 174 and SEQ ID NO: 175, SEQ ID NO: 176 and SEQ ID NO: 177, SEQ ID NO: 178 and SEQ ID NO: 179, SEQ ID NO: 180 and SEQ ID NO: 181, SEQ ID NO: 182 and SEQ ID NO: 183, SEQ ID NO: 184 and SEQ ID NO: 185, SEQ ID NO: 186 and SEQ ID NO: 187, SEQ ID NO: 188 and SEQ ID NO: 189, SEQ ID NO: 190 and SEQ ID NO: 191, and SEQ ID NO: 192 and SEQ ID NO: 193.

Advantageously, the first G/C-rich domain of the first molecule (or second molecule) corresponds to the following sequence GG CGATCGC (SEQ ID NO: 194) so that the second G/C-rich domain is the same. Indeed, due to the folding of the molecule onto itself, the second G/C-rich domain is in a complementary and antiparallel orientation with respect to the first G/C-rich domain, and the interaction takes place at the palindromic region (underlined in the sequence hereinbefore).

The first and second G/C-rich domains may also be the following sequence GCGGCGATCGGC (SEQ ID NO: 195). The explanations hereinbefore apply mutatis mutandis.

Other sequences of the G/C-rich domains of the first or second molecule may be as follows:

-first G/C-rich domain of sequence
(SEQ ID NO: 196)
GGTCGC
and
the second G/C-rich domain of sequence
(SEQ ID NO: 197)
GCGACC.

These examples are given only by way of illustration and cannot limit the scope of the invention.

In one advantageous embodiment, the A/T-rich sequences of the first molecule of said complex consist essentially, or are made up of A or T.

Even more advantageously, the A/T-rich sequence of the first molecule of said complex consists of T.

Advantageously, the invention relates to the aforementioned composition wherein the gene coding for IL2-Rγ comprises the sequence SEQ ID NO: 1 or the sequence SEQ ID NO: 2 or the sequence SEQ ID NO: 3.

The sequence SEQ ID NO: 1 corresponds to the complete human IL2Rγ gene, referenced under GenBank number: AY692262.1. This gene is also known by the following names: P64, CIDX, IMD4, CD132, SCIDX, IL-2RG and SCIDX1. It makes 7130 bases.

The exons of the gene correspond to sequences delimited as follows:
Exon 1: from 1956 (first nucleotide of the ATG initiator codon) to 2070, and intron 1: from 2071 to 2448,
Exon 2: from 2449 to 2602, and intron 2: from 2603 to 2810,
Exon 3: from 2811 to 2995, and intron 3: from 2996 to 3203, Exon 4: from 3204 to 3343, and intron 4: from 3344 to 4108,
Exon 5: from 4109 to 4271, and intron 5: from 4272 to 4803,
Exon 6: from 4804 to 4900, and intron 6: from 4901 to 5152,
Exon 7: from 5153 to 5222, and intron 7: from 5223 to 5577, and
Exon 8: from 5578 to 5763 (last nucleotide of the stop codon),
The sequence SEQ ID NO: 2 corresponds to a fragment of SEQ ID NO: 1, since its sequence starts at position 1956 of sequence SEQ ID NO: 1 and ends at the stop codon at position 5763 of SEQ ID NO: 1.

The sequence SEQ ID NO: 3 corresponds to the coding region (or CDS) of the gene. It is the one corresponding to the combination of the above-mentioned exons. This sequence is referenced under the GenBank number: AK314932.1.

In view of the above-mentioned breakdown, the person skilled in the art may choose a part of the gene as defined above, in order to achieve a partial substitution of the gene. For example, and without limitation, if it is desired to replace exon 1, it will be possible to target intron 1 with the first molecule, intron 2 with the second molecule, while the third molecule will comprise the exon 2 sequence.

Even more advantageously, the invention relates to the composition as defined above, where the first, second and third molecules are chosen from the triplets as defined in Table 2 below.

Advantageously, the invention relates to a composition comprising one of the triplets of first, second and third molecules described in the following table 2

TABLE 2

| | First molecule | Second molecule | Third molecule |
|---|---|---|---|
| Triplet | SEQ ID NO: | SEQ ID NO: | SEQ ID NO: |
| #1 | 198 | 199 | 200 |
| #2 | 201 | 202 | 203 |
| #3 | 204 | 205 | 206 |
| #4 | 207 | 208 | 209 |
| #5 | 210 | 211 | 212 |
| #6 | 213 | 214 | 215 |
| #7 | 216 | 217 | 218 |
| #8 | 219 | 220 | 221 |
| #9 | 222 | 223 | 224 |
| #10 | 225 | 226 | 227 |
| #11 | 228 | 229 | 230 |
| #12 | 231 | 232 | 233 |
| #13 | 234 | 235 | 236 |
| #14 | 237 | 238 | 239 |
| #15 | 240 | 241 | 242 |
| #16 | 243 | 244 | 245 |
| #17 | 246 | 247 | 248 |
| #18 | 249 | 250 | 251 |
| #19 | 252 | 253 | 254 |
| #20 | 255 | 256 | 257 |
| #21 | 258 | 259 | 260 |
| #22 | 261 | 262 | 263 |
| #23 | 264 | 265 | 266 |
| #24 | 267 | 268 | 269 |
| #25 | 270 | 271 | 272 |
| #26 | 273 | 274 | 275 |
| #27 | 276 | 277 | 278 |
| #28 | 279 | 280 | 281 |
| #29 | 282 | 283 | 284 |
| #30 | 285 | 286 | 287 |
| #31 | 288 | 289 | 290 |
| #32 | 291 | 292 | 293 |
| #33 | 294 | 295 | 296 |
| #34 | 297 | 298 | 299 |
| #35 | 300 | 301 | 302 |
| #36 | 303 | 304 | 305 |
| #37 | 306 | 307 | 308 |
| #38 | 309 | 310 | 311 |
| #39 | 312 | 313 | 314 |
| #40 | 315 | 316 | 317 |
| #41 | 318 | 319 | 320 |
| #42 | 321 | 322 | 323 |
| #43 | 324 | 325 | 326 |
| #44 | 327 | 328 | 329 |
| #45 | 330 | 331 | 332 |
| #46 | 333 | 334 | 335 |
| #47 | 336 | 337 | 338 |
| #48 | 339 | 340 | 341 |
| #49 | 342 | 343 | 344 |
| #50 | 345 | 346 | 347 |
| #51 | 348 | 349 | 350 |
| #52 | 351 | 352 | 353 |
| #53 | 354 | 355 | 356 |
| #54 | 357 | 358 | 359 |
| #55 | 360 | 361 | 362 |
| #56 | 363 | 364 | 365 |
| #57 | 366 | 367 | 368 |
| #58 | 369 | 370 | 371 |
| #59 | 372 | 373 | 374 |
| #60 | 375 | 376 | 377 |
| #61 | 378 | 379 | 380 |
| #62 | 381 | 382 | 383 |
| #63 | 384 | 385 | 386 |
| #64 | 387 | 388 | 389 |
| #65 | 390 | 391 | 392 |
| #66 | 393 | 394 | 395 |
| #67 | 396 | 397 | 398 |
| #68 | 399 | 400 | 401 |
| #69 | 402 | 403 | 404 |
| #70 | 405 | 406 | 407 |
| #71 | 408 | 409 | 410 |
| #72 | 411 | 412 | 413 |
| #73 | 414 | 415 | 416 |
| #74 | 417 | 418 | 419 |
| #75 | 420 | 421 | 422 |
| #76 | 423 | 424 | 425 |
| #77 | 426 | 427 | 428 |
| #78 | 429 | 430 | 431 |
| #79 | 432 | 433 | 434 |
| #80 | 435 | 436 | 437 |
| #81 | 438 | 439 | 440 |
| #82 | 441 | 442 | 443 |
| #83 | 444 | 445 | 446 |
| #84 | 447 | 448 | 449 |
| #85 | 450 | 451 | 452 |
| #86 | 453 | 454 | 455 |
| #87 | 456 | 457 | 458 |
| #88 | 459 | 460 | 461 |
| #89 | 462 | 463 | 464 |
| #90 | 465 | 466 | 467 |
| #91 | 468 | 469 | 470 |
| #92 | 471 | 472 | 473 |
| #93 | 474 | 475 | 476 |
| #94 | 477 | 478 | 479 |
| #95 | 480 | 481 | 482 |
| #96 | 483 | 484 | 485 |
| #97 | 486 | 487 | 488 |
| #98 | 489 | 490 | 491 |
| #99 | 492 | 493 | 494 |
| #100 | 495 | 496 | 497 |
| #101 | 498 | 499 | 500 |
| #102 | 501 | 502 | 503 |
| #103 | 504 | 505 | 506 |
| #104 | 507 | 508 | 509 |
| #105 | 510 | 511 | 512 |
| #106 | 513 | 514 | 515 |
| #107 | 516 | 517 | 518 |
| #108 | 519 | 520 | 521 |
| #109 | 522 | 523 | 524 |
| #110 | 525 | 526 | 527 |
| #111 | 528 | 529 | 530 |
| #112 | 531 | 532 | 533 |
| #113 | 534 | 535 | 536 |
| #114 | 537 | 538 | 539 |
| #115 | 540 | 541 | 542 |
| #116 | 543 | 544 | 545 |
| #117 | 546 | 547 | 548 |
| #118 | 549 | 550 | 551 |
| #119 | 552 | 553 | 554 |
| #120 | 555 | 556 | 557 |
| #121 | 558 | 559 | 560 |
| #122 | 561 | 562 | 563 |
| #123 | 564 | 565 | 566 |
| #124 | 567 | 568 | 569 |
| #125 | 570 | 571 | 572 |
| #126 | 573 | 574 | 575 |
| #127 | 576 | 577 | 578 |
| #128 | 579 | 580 | 581 |
| #129 | 582 | 583 | 584 |
| #130 | 585 | 586 | 587 |
| #131 | 588 | 589 | 590 |
| #132 | 591 | 592 | 593 |
| #133 | 594 | 595 | 596 |
| #134 | 597 | 598 | 599 |
| #135 | 600 | 601 | 602 |
| #136 | 603 | 604 | 605 |
| #137 | 606 | 607 | 608 |
| #138 | 609 | 610 | 611 |
| #139 | 612 | 613 | 614 |
| #140 | 615 | 616 | 617 |
| #141 | 618 | 619 | 620 |
| #142 | 621 | 622 | 623 |
| #143 | 624 | 625 | 626 |
| #144 | 627 | 628 | 629 |
| #145 | 630 | 631 | 632 |
| #146 | 633 | 634 | 635 |
| #147 | 636 | 637 | 638 |
| #148 | 639 | 640 | 641 |
| #149 | 642 | 643 | 644 |
| #150 | 645 | 646 | 647 |
| #151 | 648 | 649 | 650 |
| #152 | 651 | 652 | 653 |
| #153 | 654 | 655 | 656 |
| #154 | 657 | 658 | 659 |
| #155 | 660 | 661 | 662 |
| #156 | 663 | 664 | 665 |
| #157 | 666 | 667 | 668 |
| #158 | 669 | 670 | 671 |
| #159 | 672 | 673 | 674 |
| #160 | 675 | 676 | 677 |
| #161 | 678 | 679 | 680 |
| #162 | 681 | 682 | 683 |
| #163 | 684 | 685 | 686 |
| #164 | 687 | 688 | 689 |
| #165 | 690 | 691 | 692 |
| #166 | 693 | 694 | 695 |
| #167 | 696 | 697 | 698 |
| #168 | 699 | 700 | 701 |
| #169 | 702 | 703 | 704 |
| #170 | 705 | 706 | 707 |
| #171 | 708 | 709 | 710 |
| #172 | 711 | 712 | 713 |
| #173 | 714 | 715 | 716 |
| #174 | 717 | 718 | 719 |
| #175 | 720 | 721 | 722 |
| #176 | 723 | 724 | 725 |
| #177 | 726 | 727 | 728 |
| #178 | 729 | 730 | 731 |
| #179 | 732 | 733 | 734 |
| #180 | 735 | 736 | 737 |
| #181 | 738 | 739 | 740 |
| #182 | 741 | 742 | 743 |
| #183 | 744 | 745 | 746 |
| #184 | 747 | 748 | 749 |
| #185 | 750 | 751 | 752 |
| #186 | 753 | 754 | 755 |
| #187 | 756 | 757 | 758 |
| #188 | 759 | 760 | 761 |
| #189 | 762 | 763 | 764 |
| #190 | 765 | 766 | 767 |
| #191 | 768 | 769 | 770 |
| #192 | 771 | 772 | 773 |
| #193 | 774 | 775 | 776 |
| #194 | 777 | 778 | 779 |
| #195 | 780 | 781 | 782 |
| #196 | 783 | 784 | 785 |
| #197 | 786 | 787 | 788 |
| #198 | 789 | 790 | 791 |
| #199 | 792 | 793 | 794 |
| #200 | 795 | 796 | 797 |
| #201 | 798 | 799 | 800 |
| #202 | 801 | 802 | 803 |
| #203 | 804 | 805 | 806 |
| #204 | 807 | 808 | 809 |
| #205 | 810 | 811 | 812 |
| #206 | 813 | 814 | 815 |
| #207 | 816 | 817 | 818 |
| #208 | 819 | 820 | 821 |
| #209 | 822 | 823 | 824 |
| #210 | 825 | 826 | 827 |
| #211 | 828 | 829 | 830 |
| #212 | 831 | 832 | 833 |
| #213 | 834 | 835 | 836 |
| #214 | 837 | 838 | 839 |
| #215 | 840 | 841 | 842 |
| #216 | 843 | 844 | 845 |
| #217 | 846 | 847 | 848 |
| #218 | 849 | 850 | 851 |
| #219 | 852 | 853 | 854 |
| #220 | 855 | 856 | 857 |
| #221 | 858 | 859 | 860 |
| #222 | 861 | 862 | 863 |
| #223 | 864 | 865 | 866 |
| #224 | 867 | 868 | 869 |
| #225 | 870 | 871 | 872 |
| #226 | 873 | 874 | 875 |
| #227 | 876 | 877 | 878 |
| #228 | 879 | 880 | 881 |
| #229 | 882 | 883 | 884 |
| #230 | 885 | 886 | 887 |
| #231 | 888 | 889 | 890 |
| #232 | 891 | 892 | 893 |
| #233 | 894 | 895 | 896 |
| #234 | 897 | 898 | 899 |
| #235 | 900 | 901 | 902 |
| #236 | 903 | 904 | 905 |
| #237 | 906 | 907 | 908 |
| #238 | 909 | 910 | 911 |
| #239 | 912 | 913 | 914 |
| #240 | 915 | 916 | 917 |
| #241 | 918 | 919 | 920 |
| #242 | 921 | 922 | 923 |
| #243 | 924 | 925 | 926 |
| #244 | 927 | 928 | 929 |
| #245 | 930 | 931 | 932 |
| #246 | 933 | 934 | 935 |
| #247 | 936 | 937 | 938 |
| #248 | 939 | 940 | 941 |
| #249 | 942 | 943 | 944 |
| #250 | 945 | 946 | 947 |
| #251 | 948 | 949 | 950 |
| #252 | 951 | 952 | 953 |
| #253 | 954 | 955 | 956 |
| #254 | 957 | 958 | 959 |
| #255 | 960 | 961 | 962 |
| #256 | 963 | 964 | 965 |
| #257 | 966 | 967 | 968 |
| #258 | 969 | 970 | 971 |
| #259 | 972 | 973 | 974 |
| #260 | 975 | 976 | 977 |
| #261 | 978 | 979 | 980 |
| #262 | 981 | 982 | 983 |
| #263 | 984 | 985 | 986 |
| #264 | 987 | 988 | 989 |
| #265 | 990 | 991 | 992 |
| #266 | 993 | 994 | 995 |
| #267 | 996 | 997 | 998 |
| #268 | 999 | 1000 | 1001 |
| #269 | 1002 | 1003 | 1004 |
| #270 | 1005 | 1006 | 1007 |
| #271 | 1008 | 1009 | 1010 |
| #272 | 1011 | 1012 | 1013 |
| #273 | 1014 | 1015 | 1016 |
| #274 | 1017 | 1018 | 1019 |
| #275 | 1020 | 1021 | 1022 |
| #276 | 1023 | 1024 | 1025 |
| #277 | 1026 | 1027 | 1028 |
| #278 | 1029 | 1030 | 1031 |
| #279 | 1032 | 1033 | 1034 |
| #280 | 1035 | 1036 | 1037 |
| #281 | 1038 | 1039 | 1040 |
| #282 | 1041 | 1042 | 1043 |
| #283 | 1044 | 1045 | 1046 |
| #284 | 1047 | 1048 | 1049 |
| #285 | 1050 | 1051 | 1052 |
| #286 | 1053 | 1054 | 1055 |
| #287 | 1056 | 1057 | 1058 |
| #288 | 1059 | 1060 | 1061 |
| #289 | 1062 | 1063 | 1064 |
| #290 | 1065 | 1066 | 1067 |
| #291 | 1068 | 1069 | 1070 |
| #292 | 1071 | 1072 | 1073 |
| #293 | 1074 | 1075 | 1076 |
| #294 | 1077 | 1078 | 1079 |
| #295 | 1080 | 1081 | 1082 |
| #296 | 1083 | 1084 | 1085 |
| #297 | 1086 | 1087 | 1088 |
| #298 | 1089 | 1090 | 1091 |
| #299 | 1092 | 1093 | 1094 |
| #300 | 1095 | 1096 | 1097 |
| #301 | 1098 | 1099 | 1100 |
| #302 | 1101 | 1102 | 1103 |
| #303 | 1104 | 1105 | 1106 |
| #304 | 1107 | 1108 | 1109 |
| #305 | 1110 | 1111 | 1112 |
| #306 | 1113 | 1114 | 1115 |
| #307 | 1116 | 1117 | 1118 |
| #308 | 1119 | 1120 | 1121 |
| #309 | 1122 | 1123 | 1124 |
| #310 | 1125 | 1126 | 1127 |
| #311 | 1128 | 1129 | 1130 |
| #312 | 1131 | 1132 | 1133 |

Also advantageously, the invention relates to the above-mentioned composition, said composition comprising a triplet selected from the above-mentioned triplets #1 to #312.

Advantageously, the invention relates to the composition described above, further comprising
a fourth single-stranded nucleic acid molecule comprising or consisting essentially of an A′sequence allowing the insertion of a complementary sequence of a nucleic acid of interest, or comprising a complementary sequence of a nucleic acid of interest, said complementary sequence binding at 5′ to a fifth T-rich sequence of 40 to 60 nucleotides in length and at 3′ to a sixth T-rich sequence of 40 to 60 nucleotides in length, said fifth and sixth T-rich sequences respectively comprising a fifth and a sixth domain of 6 to 12 G/C-rich nucleotides, the sequence of the fifth domain being complementary to the sequence of the sixth domain, said fifth and sixth domains being positioned 15 to 52 nucleotides from said A′ sequence, said fourth molecule comprising at its 5′ end at least one first sequence oriented 5′-to-3′ for recognizing a transposase and at its 3′ end a second sequence for recognizing said transposase,
a fifth single-stranded nucleic acid molecule comprising or consisting essentially of a B′sequence allowing the insertion of a complementary sequence of a nucleic acid of interest, or comprising a complementary sequence of a nucleic acid of interest, said complementary B′ sequence binding at 5′ to a seventh T-rich sequence of 40 to 60 nucleotides in length and at 3′ to an eighth T-rich sequence of 40 to 60 nucleotides in length, said seventh and eighth T-rich sequences respectively comprising a third and a fourth domain of 6 to 12 G-C-rich nucleotides, the sequence of the seventh domain being complementary to the sequence of the eighth domain, said seventh and eighth domains being positioned 15 to 52 nucleotides from said B′ sequence, said fifth molecule comprising at its 5′ end at least one first sequence oriented 5′-to-3′ for recognizing said transposase and at its 3′ end the second sequence for recognizing said transposase,

said B′ sequence being a complementary sequence of said nucleic acid of interest, the A′ sequence being positioned at 5′ of a region of interest of said nucleic acid of interest and the B′ sequence positioned at 3′ of the region of interest of said nucleic acid of interest, and
a sixth single-stranded molecule comprising
in its 5′ part, at least one complementary sequence of said fifth sequence for recognizing said transposase of the first molecule,
in its 3′ part, at least one complementary sequence of said fourth sequence for recognizing said transposase of the second molecule, and
an intermediate region located between the complementary sequence of said fourth recognition sequence of said transposase of the first molecule and the complementary sequence of said fifth recognition sequence of said transposase of the second molecule, said intermediate region comprising a sequence that is antiparallel and complementary to the sequence coding for the interleukin-2 receptor gamma or IL2-Rγ contained in the third molecule of the first composition,

the fourth and sixth single-stranded nucleic acid molecules being paired according to the base complementarity defined by Watson and Crick so as to define two double-stranded binding sites of said transposase, and the fifth and sixth single-stranded nucleic acid molecules being paired according to the base complementarity defined by Watson and Crick so as to define two double-stranded binding sites of said transposase.

Advantageously, the composition according to the invention comprises 6 molecules, that is, two triplets enabling double-stranded replacement of a target molecule. This replacement can only take place if the regions A, B and A′ and B′ are correctly chosen, so that the region of interest to be replaced is correctly framed.

Of course, all the definitions given for a 3-molecule composition apply mutatis mutandis to a 6-molecule composition.

Advantageously, the invention relates to the composition described above, wherein
said A sequence comprises a sequence complementary to the sequence of a first region of the gene coding for IL2-Rγ,
said B sequence comprises a sequence complementary to the sequence of a second region of the gene coding for IL2-Rγ,

said A sequence and said B sequence being two different sequences, said first and second regions of the gene coding for IL2-Rγ, framing a region comprising a sequence coding for a portion of IL2-Rγ, or coding for the complete IL2-Rγ receptor, wherein
said A′ sequence comprises a sequence complementary to the sequence of a third region of the gene coding for IL2-Rγ,
said B′ sequence comprises a sequence complementary to the sequence of a fourth region of the gene coding for IL2-Rγ,

said A′ sequence and said B′ sequence being two different sequences, said third and fourth regions of the gene coding for IL2-Rγ, framing a region comprising a sequence coding for a portion of IL2-Rγ, or coding for the complete IL2-Rγ receptor,

and wherein

the A sequence and the A′ sequence are at most partially complementary,

the A sequence and the B′ sequence are at most partially complementary,

the B sequence and the A′ sequence are at most partially complementary, and

the B sequence and the B′ sequence are at most partially complementary.

“At most partially complementary” within the invention means that the sequences possess sequences that are capable of pairing according to the base complementarity defined by Watson and Crick, but not on all of the sequences. In other words, only part of the sequence is complementary. Advantageously, the sequences A, A′, B and B′ are partially complementary on less than 50% of the sequences, in particular less than 30% of the sequences, in particular less than 10% of the sequences, in particular are not complementary to each other at all.

In this way, it is possible to frame the gene coding for the IL2-Rγ receptor in a specific and oriented way, for both strands, in order to avoid disordered or inconsistent recombinations (that is, tagmentations) so that the result would not be as expected.

Even more advantageously, the above-mentioned composition comprises at least one of the sextuplets mentioned in the following Table 3:

TABLE 3

| | First | Second | Third | Fifth | Fourth | Sixth |
|---|---|---|---|---|---|---|
| | molecule | molecule | molecule | molecule | molecule | molecule |
| | SEQ | SEQ | SEQ | SEQ | SEQ | SEQ |
| Sextuplet | ID NO: | ID NO: | ID NO: | ID NO: | ID NO: | ID NO: |
| #1 | 198 | 199 | 200 | 201 | 202 | 203 |
| #2 | 204 | 205 | 206 | 207 | 208 | 209 |
| #3 | 210 | 211 | 212 | 213 | 214 | 215 |
| #4 | 216 | 217 | 218 | 219 | 220 | 221 |
| #5 | 222 | 223 | 224 | 225 | 226 | 227 |
| #6 | 228 | 229 | 230 | 231 | 232 | 233 |
| #7 | 234 | 235 | 236 | 237 | 238 | 239 |
| #8 | 240 | 241 | 242 | 243 | 244 | 245 |
| #9 | 246 | 247 | 248 | 249 | 250 | 251 |
| #10 | 252 | 253 | 254 | 255 | 256 | 257 |
| #11 | 258 | 259 | 260 | 261 | 262 | 263 |
| #12 | 264 | 265 | 266 | 267 | 268 | 269 |
| #13 | 270 | 271 | 272 | 273 | 274 | 275 |
| #14 | 276 | 277 | 278 | 279 | 280 | 281 |
| #15 | 282 | 283 | 284 | 285 | 286 | 287 |
| #16 | 288 | 289 | 290 | 291 | 292 | 293 |
| #17 | 294 | 295 | 296 | 297 | 298 | 299 |
| #18 | 300 | 301 | 302 | 303 | 304 | 305 |
| #19 | 306 | 307 | 308 | 309 | 310 | 311 |
| #20 | 312 | 313 | 314 | 315 | 316 | 317 |
| #21 | 318 | 319 | 320 | 321 | 322 | 323 |
| #22 | 324 | 325 | 326 | 327 | 328 | 329 |
| #23 | 330 | 331 | 332 | 333 | 334 | 335 |
| #24 | 336 | 337 | 338 | 339 | 340 | 341 |
| #25 | 342 | 343 | 344 | 345 | 346 | 347 |
| #26 | 348 | 349 | 350 | 351 | 352 | 353 |
| #27 | 354 | 355 | 356 | 357 | 358 | 359 |
| #28 | 360 | 361 | 362 | 363 | 364 | 365 |
| #29 | 366 | 367 | 368 | 369 | 370 | 371 |
| #30 | 372 | 373 | 374 | 375 | 376 | 377 |
| #31 | 378 | 379 | 380 | 381 | 382 | 383 |
| #32 | 384 | 385 | 386 | 387 | 388 | 389 |
| #33 | 390 | 391 | 392 | 393 | 394 | 395 |
| #34 | 396 | 397 | 398 | 399 | 400 | 401 |
| #35 | 402 | 403 | 404 | 405 | 406 | 407 |
| #36 | 408 | 409 | 410 | 411 | 412 | 413 |
| #37 | 414 | 415 | 416 | 417 | 418 | 419 |
| #38 | 420 | 421 | 422 | 423 | 424 | 425 |
| #39 | 426 | 427 | 428 | 429 | 430 | 431 |
| #40 | 432 | 433 | 434 | 435 | 436 | 437 |
| #41 | 438 | 439 | 440 | 441 | 442 | 443 |
| #42 | 444 | 445 | 446 | 447 | 448 | 449 |
| #43 | 450 | 451 | 452 | 453 | 454 | 455 |
| #44 | 456 | 457 | 458 | 459 | 460 | 461 |
| #45 | 462 | 463 | 464 | 465 | 466 | 467 |
| #46 | 468 | 469 | 470 | 471 | 472 | 473 |
| #47 | 474 | 475 | 476 | 477 | 478 | 479 |
| #48 | 480 | 481 | 482 | 483 | 484 | 485 |
| #49 | 486 | 487 | 488 | 489 | 490 | 491 |
| #50 | 492 | 493 | 494 | 495 | 496 | 497 |
| #51 | 498 | 499 | 500 | 501 | 502 | 503 |
| #52 | 504 | 505 | 506 | 507 | 508 | 509 |
| #53 | 510 | 511 | 512 | 513 | 514 | 515 |
| #54 | 516 | 517 | 518 | 519 | 520 | 521 |
| #55 | 522 | 523 | 524 | 525 | 526 | 527 |
| #56 | 528 | 529 | 530 | 531 | 532 | 533 |
| #57 | 534 | 535 | 536 | 537 | 538 | 539 |
| #58 | 540 | 541 | 542 | 543 | 544 | 545 |
| #59 | 546 | 547 | 548 | 549 | 550 | 551 |
| #60 | 552 | 553 | 554 | 555 | 556 | 557 |
| #61 | 558 | 559 | 560 | 561 | 562 | 563 |
| #62 | 564 | 565 | 566 | 567 | 568 | 569 |
| #63 | 570 | 571 | 572 | 573 | 574 | 575 |
| #64 | 576 | 577 | 578 | 579 | 580 | 581 |
| #65 | 582 | 583 | 584 | 585 | 586 | 587 |
| #66 | 588 | 589 | 590 | 591 | 592 | 593 |
| #67 | 594 | 595 | 596 | 597 | 598 | 599 |
| #68 | 600 | 601 | 602 | 603 | 604 | 605 |
| #69 | 606 | 607 | 608 | 609 | 610 | 611 |
| #70 | 612 | 613 | 614 | 615 | 616 | 617 |
| #71 | 618 | 619 | 620 | 621 | 622 | 623 |
| #72 | 624 | 625 | 626 | 627 | 628 | 629 |
| #73 | 630 | 631 | 632 | 633 | 634 | 635 |
| #74 | 636 | 637 | 638 | 639 | 640 | 641 |
| #75 | 642 | 643 | 644 | 645 | 646 | 647 |
| #76 | 648 | 649 | 650 | 651 | 652 | 653 |
| #77 | 654 | 655 | 656 | 657 | 658 | 659 |
| #78 | 660 | 661 | 662 | 663 | 664 | 665 |
| #79 | 666 | 667 | 668 | 669 | 670 | 671 |
| #80 | 672 | 673 | 674 | 675 | 676 | 677 |
| #81 | 678 | 679 | 680 | 681 | 682 | 683 |
| #82 | 684 | 685 | 686 | 687 | 688 | 689 |
| #83 | 690 | 691 | 692 | 693 | 694 | 695 |
| #84 | 696 | 697 | 698 | 699 | 700 | 701 |
| #85 | 702 | 703 | 704 | 705 | 706 | 707 |
| #86 | 708 | 709 | 710 | 711 | 712 | 713 |
| #87 | 714 | 715 | 716 | 717 | 718 | 719 |
| #88 | 720 | 721 | 722 | 723 | 724 | 725 |
| #89 | 726 | 727 | 728 | 729 | 730 | 731 |
| #90 | 732 | 733 | 734 | 735 | 736 | 737 |
| #91 | 738 | 739 | 740 | 741 | 742 | 743 |
| #92 | 744 | 745 | 746 | 747 | 748 | 749 |
| #93 | 750 | 751 | 752 | 753 | 754 | 755 |
| #94 | 756 | 757 | 758 | 759 | 760 | 761 |
| #95 | 762 | 763 | 764 | 765 | 766 | 767 |
| #96 | 768 | 769 | 770 | 771 | 772 | 773 |
| #97 | 774 | 775 | 776 | 777 | 778 | 779 |
| #98 | 780 | 781 | 782 | 783 | 784 | 785 |
| #99 | 786 | 787 | 788 | 789 | 790 | 791 |
| #100 | 792 | 793 | 794 | 795 | 796 | 797 |
| #101 | 798 | 799 | 800 | 801 | 802 | 803 |
| #102 | 804 | 805 | 806 | 807 | 808 | 809 |
| #103 | 810 | 811 | 812 | 813 | 814 | 815 |
| #104 | 816 | 817 | 818 | 819 | 820 | 821 |
| #105 | 822 | 823 | 824 | 825 | 826 | 827 |
| #106 | 828 | 829 | 830 | 831 | 832 | 833 |
| #107 | 834 | 835 | 836 | 837 | 838 | 839 |
| #108 | 840 | 841 | 842 | 843 | 844 | 845 |
| #109 | 846 | 847 | 848 | 849 | 850 | 851 |
| #110 | 852 | 853 | 854 | 855 | 856 | 857 |
| #111 | 858 | 859 | 860 | 861 | 862 | 863 |
| #112 | 864 | 865 | 866 | 867 | 868 | 869 |
| #113 | 870 | 871 | 872 | 873 | 874 | 875 |
| #114 | 876 | 877 | 878 | 879 | 880 | 881 |
| #115 | 882 | 883 | 884 | 885 | 886 | 887 |
| #116 | 888 | 889 | 890 | 891 | 892 | 893 |
| #117 | 894 | 895 | 896 | 897 | 898 | 899 |
| #118 | 900 | 901 | 902 | 903 | 904 | 905 |
| #119 | 906 | 907 | 908 | 909 | 910 | 911 |
| #120 | 912 | 913 | 914 | 915 | 916 | 917 |
| #121 | 918 | 919 | 920 | 921 | 922 | 923 |
| #122 | 924 | 925 | 926 | 927 | 928 | 929 |
| #123 | 930 | 931 | 932 | 933 | 934 | 935 |
| #124 | 936 | 937 | 938 | 939 | 940 | 941 |
| #125 | 942 | 943 | 944 | 945 | 946 | 947 |
| #126 | 948 | 949 | 950 | 951 | 952 | 953 |
| #127 | 954 | 955 | 956 | 957 | 958 | 959 |
| #128 | 960 | 961 | 962 | 963 | 964 | 965 |
| #129 | 966 | 967 | 968 | 969 | 970 | 971 |
| #130 | 972 | 973 | 974 | 975 | 976 | 977 |
| #131 | 978 | 979 | 980 | 981 | 982 | 983 |
| #132 | 984 | 985 | 986 | 987 | 988 | 989 |
| #133 | 990 | 991 | 992 | 993 | 994 | 995 |
| #134 | 996 | 997 | 998 | 999 | 1000 | 1001 |
| #135 | 1002 | 1003 | 1004 | 1005 | 1006 | 1007 |
| #136 | 1008 | 1009 | 1010 | 1011 | 1012 | 1013 |
| #137 | 1014 | 1015 | 1016 | 1017 | 1018 | 1019 |
| #138 | 1020 | 1021 | 1022 | 1023 | 1024 | 1025 |
| #139 | 1026 | 1027 | 1028 | 1029 | 1030 | 1031 |
| #140 | 1032 | 1033 | 1034 | 1035 | 1036 | 1037 |
| #141 | 1038 | 1039 | 1040 | 1041 | 1042 | 1043 |
| #142 | 1044 | 1045 | 1046 | 1047 | 1048 | 1049 |
| #143 | 1050 | 1051 | 1052 | 1053 | 1054 | 1055 |
| #144 | 1056 | 1057 | 1058 | 1059 | 1060 | 1061 |
| #145 | 1062 | 1063 | 1064 | 1065 | 1066 | 1067 |
| #146 | 1068 | 1069 | 1070 | 1071 | 1072 | 1073 |
| #147 | 1074 | 1075 | 1076 | 1077 | 1078 | 1079 |
| #148 | 1080 | 1081 | 1082 | 1083 | 1084 | 1085 |
| #149 | 1086 | 1087 | 1088 | 1089 | 1090 | 1091 |
| #150 | 1092 | 1093 | 1094 | 1095 | 1096 | 1097 |
| #151 | 1098 | 1099 | 1100 | 1101 | 1102 | 1103 |
| #152 | 1104 | 1105 | 1106 | 1107 | 1108 | 1109 |
| #153 | 1110 | 1111 | 1112 | 1113 | 1114 | 1115 |
| #154 | 1116 | 1117 | 1118 | 1119 | 1120 | 1121 |
| #155 | 1122 | 1123 | 1124 | 1125 | 1126 | 1127 |
| #156 | 1128 | 1129 | 1130 | 1131 | 1132 | 1133 |

The composition according to the invention thus advantageously offers 156 sextuplets of molecules enabling a potentially mutated or abnormal sequence to be replaced by the reference wild-type sequence at the locus of the IL2R geneγ.

Depending on the target cell, this makes it possible to restore a function lost through mutation, and to achieve functional complementation.

In another aspect, the invention relates to a pharmaceutical composition comprising the above-mentioned composition, in association with a pharmaceutically acceptable vehicle.

The above-mentioned composition, in association with a pharmaceutically acceptable vehicle, can be used in the treatment of pathologies. The vehicle is a commonly accepted vehicle known to the person skilled in the art, such as distilled water or a physiological buffer. This vehicle must allow the formation of a complex as mentioned above, but also be acceptable to a cell or living being.

In another aspect, the invention relates to the above-mentioned composition, for use as a medicament.

Moreover, it is advantageous for the composition comprising any of the triplets in Table 2, or any of the sextuplets in Table 3, to be used as a medicament.

In yet another aspect, the invention concerns a composition for use in the treatment of pathologies linked to a mutation in the gene coding for IL2-Rγ.

Mutations in the IL2Rγ gene are widely described in the literature, and may correspond to substitutions, insertions or deletions affecting either an exon, an intron or the 5′ or 3′ regulatory regions of the gene.

More particularly, the invention concerns a composition for use in the treatment of pathologies linked to a mutation in the gene coding for IL2-Rγ, said composition comprising a triplet as mentioned in table 2 or a sextuplet as mentioned in table 3.

Advantageously, the invention relates to the composition for its aforementioned use, where the disease associated with a mutation in the gene coding for IL2-Rγ is SCID-X disease or Omenn syndrome.

Severe combined immunodeficiency (SCID) T-B+ by gamma chain deficiency, also known as SCIDX1 or SCID-X, is a form of SCID characterized by severe and recurrent infections associated with diarrhea and stunted growth.

During the first months of life, SCIDX1 manifests itself in severe and often fatal viral, bacterial or fungal infections (e.g. Pneumocystis jiroveci pneumonitis, disseminated BCG infection secondary to vaccination) and stunted growth. Chronic diarrhea is a frequent feature. Some patients experience skin rashes and liver function abnormalities. Graft-versus-host disease, linked to maternal-fetal transmission, is also associated with the disease. Immunological results reveal lymphopenia with absence of T or NK lymphocytes, hypogammaglobulinemia, and normal or elevated B lymphocyte counts.

Omenn syndrome is an inflammatory disorder characterized by erythroderma, desquamation, alopecia, chronic diarrhea, staturo-ponderal growth retardation, adenopathy and hepatosplenomegaly, associated with severe combined immunodeficiency. Omenn syndrome appears in the first year of life, with manifestations characteristic of severe combined immunodeficiency, including chronic diarrhea, pneumonitis and staturo-ponderal growth retardation. Patients also present inflammatory symptoms such as adenopathy, hepatosplenomegaly and generalized erythroderma, often leading to alopecia and loss of eyebrows and eyelashes. Protein loss can lead to generalized edema and metabolic disorders. Signs and symptoms may evolve over time and may appear separately. Some patients manifest only certain symptoms, and can be described as cases of Atypical Omenn Syndrome. Rather than a distinct form of severe combined immunodeficiency, it is more an inflammatory phenotype that can be associated with different types of severe combined immunodeficiency. Most cases reported to date feature hypomorphic mutations in the RAG1 and RAG2 (11p13) genes. Other mutations occur in the RMRP, ADA and IL2Rγ genes, as well as others.

In another aspect, the invention relates to a method of treating SCID-X or Omenn syndrome, said method comprising administering an effective amount of an aforementioned composition to an individual in need, said composition notably comprising a triplet as defined in Table 2 or a sextuplet as defined in Table 3.

In yet another aspect, the invention relates to the use of a composition as defined above, for the substitution, in a somatic cell, of a mutated sequence of the gene coding for IL2-Rγ by a wild-type sequence of the gene coding wild-type IL2-Rγ, with the proviso that the use does not comprise a method for the modification of the germline genetic identity of human beings and that said use is not a method for the treatment of the human or animal body by surgery or therapy.

As explained hereinbefore, the aforementioned composition makes it possible to target specifically a target region and to replace it with a sequence of interest, by using the transposase tagmentation properties.

The composition according to the invention is particularly advantageous for carrying out targeted gene modifications (and in particular gene replacements, or replacements of non-coding sequences) in humans and in particular for replacing the mutated IL2Rγ gene with its wild-type reference sequence.

In yet another advantageous embodiment, the invention relates to a method of replacing, in particular in vitro, a mutated sequence of the gene coding for IL2-Rγ with a wild-type sequence coding for IL2-Rγ, said method comprising:
bringing a composition as defined above into contact with the nucleic acid comprising the mutated sequence coding for the IL2-Rγ gene,

said composition being such that

the A sequence of said first molecule comprises a complementary sequence of the region immediately at 5′ of the mutated sequence coding for the IL2-Rγ gene,

the B sequence of said second molecule comprises a complementary sequence of the region immediately at 3′ of the mutated sequence coding for the IL2-Rγ gene, and

the third molecule comprises the wild-type sequence of the gene coding for IL2-Rγ, located between a complementary sequence of said second sequence for recognizing said transposase of the first molecule and the complementary sequence of said first sequence for recognizing said transposase of the second molecule, in order to obtain a replacement complex,
placing the replacement complex in the presence of a transposase recognizing the double-stranded binding sites of said transposase contained in said ensemble, to obtain a recombination complex, and
recombining the combination complex to obtain the hybrid nucleic acid molecule the wild-type sequence of the gene coding for IL2-Rγ, in place of the mutated sequence of the gene coding for IL2-Rγ.

Advantageously, the invention relates to a method of replacing, in particular in vitro, a mutated sequence of the gene coding for IL2-Rγ with a wild-type sequence coding for IL2-Rγ, said method comprising:
bringing a composition comprising a triplet as defined in Table 2 or a sextuplet as defined in Table 3, into contact with nucleic acid comprising the mutated sequence coding for the IL2-Rγ gene, to obtain a replacement complex,
placing the replacement complex in the presence of a transposase recognizing the double-stranded binding sites of said transposase contained in said ensemble, to obtain a recombination complex, and
recombining the combination complex to obtain the hybrid nucleic acid molecule the wild-type sequence of the gene coding for IL2-Rγ, in place of the mutated sequence of the gene coding for IL2-Rγ.

In yet another aspect, the invention relates to a method for in vitro replacement of a mutated double-stranded sequence of the gene coding for IL2-Rγ of a stem cell, in particular a hematopoietic stem cell, by a wild-type double-stranded sequence coding for IL2-Rγ, said method comprising:
bringing a composition as defined above into contact with the hematopoietic stem cell comprising the mutated sequence coding for the IL2-Rγ gene,

said ensemble being such that

the A sequence of said first molecule comprises a complementary sequence of the region immediately at 5′ of the mutated sequence coding for the IL2-Rγ gene,

the A′ sequence of said fourth molecule comprises a complementary sequence of the region immediately at 5′ of the mutated complementary sequence coding for the IL2-Rγ gene,

the B sequence of said second molecule comprises a complementary sequence of the region immediately at 3′ of the mutated sequence coding for the IL2-Rγ gene, the B′ sequence of said second molecule comprises a complementary

sequence of the region immediately at 3′ of the mutated complementary sequence coding for the IL2-Rγ gene, and

the third molecule comprises the wild-type sequence of the gene coding for IL2-Rγ, located between a complementary sequence of said second sequence for recognizing said transposase of the first molecule and the complementary sequence of said first sequence for recognizing said transposase of the second molecule,

the sixth molecule comprises the sequence complementary to the wild-type sequence of the gene coding for IL2-Rγ, located between a complementary sequence of said second sequence for recognizing said transposase of the first molecule and the complementary sequence of said first sequence for recognizing said transposase of the second molecule, in order to obtain a replacement complex,
placing the replacement complex in the presence of a transposase recognizing the double-stranded binding sites of said transposase contained in said composition, to obtain a recombination complex, and
recombining the combination complex to obtain the hybrid nucleic acid molecule the wild-type sequence of the gene coding for IL2-Rγ, in place of the mutated sequence of the gene coding for IL2-Rγ,
and possibly purification of hematopoietic stem cells with mutated gene replacement.

Advantageously, the invention relates to a method for in vitro replacement of a mutated double-stranded sequence of the gene coding for IL2-Rγ of a stem cell, in particular a hematopoietic stem cell, by a wild-type double-stranded sequence coding for IL2-Rγ, said method comprising:
contacting a composition as defined above with the hematopoietic stem cell comprising the mutated sequence coding for the IL2-Rγ gene to obtain a replacement complex, said composition comprising a triplet as defined in Table 2 or a sextuplet as defined in Table 3,
placing the replacement complex in the presence of a transposase recognizing the double-stranded binding sites of said transposase contained in said composition, to obtain a recombination complex, and
recombining the combination complex to obtain the hybrid nucleic acid molecule the wild-type sequence of the gene coding for IL2-Rγ, in place of the mutated sequence of the gene coding for IL2-Rγ,
and possibly purification of hematopoietic stem cells with mutated gene replacement.

In yet another aspect, the invention relates to a method of inserting, in particular in vitro, a double-stranded sequence of the wild-type gene coding for IL2-Rγ into a cell, in particular a stem cell, in particular a hematopoietic stem cell, at the locus of the gene coding for IL2-Rγ, wherein said locus of the gene coding for IL2-Rγ comprises a mutation affecting the expression, the function, or both, of IL2-Rγ,

wherein said double-stranded sequence of the wild-type gene coding for IL2-Rγ corresponds to the cDNA of said gene, in particular the sequence SEQ ID NO: 3,

said method comprising:
brining a composition as defined in any one of claims 1 to 9 into contact with the cell comprising a mutation affecting the expression, function or both of IL2-Rγ, at the locus of the gene coding for IL2-Rγ said composition being such that

the A sequence of said first molecule comprises a complementary sequence of the region at 5′ of exon 1 of the gene coding for IL2-Rγ,

the A′ sequence of said fourth molecule comprises a complementary sequence of the complementary sequence of the region at 5′ of exon 1 of the gene coding for IL2-Rγ,

the B sequence of said second molecule comprises a complementary sequence at 3′ of the region at 5′ of exon 1 of the gene coding for IL2-Rγ,

the B′ sequence of said fifth molecule comprises a complementary sequence at 3′ of the complementary sequence of the region at 5′ of exon 1 of the gene coding for IL2-Rγ, and

the third molecule comprises the wild-type cDNA sequence of the gene coding for IL2-Rγ, located between a complementary sequence of said second sequence for recognizing said transposase of the first molecule and the complementary sequence of said first sequence for recognizing said transposase of the second molecule,

the sixth molecule comprises the wild-type cDNA sequence of the gene coding for IL2-Rγ, located between a complementary sequence of said second sequence for recognizing said transposase of the first molecule and the complementary sequence of said first sequence for recognizing said transposase of the second molecule,

to obtain a replacement complex,
placing the replacement complex in the presence of a transposase
recognizing the double-stranded binding sites of said transposase contained in said ensemble, to obtain a recombination complex, and
recombining the combination complex to obtain insertion of the cDNA of the gene coding for IL2-Rγ 5′ to exon 1 of the gene coding for IL2-Rγ,
and, if necessary, purification of the inserted cell.

### EXAMPLES

### Example 1—Obtaining B Cells Expressing the Wild-Type ILR2G Gene

In order to treat patients suffering from SCID-X, involving a mutation in the ILR2G gene, it may be advantageous to propose a cell therapy aimed at transplanting into patients CD34+ hematopoietic stem cells that have been modified using a composition according to the invention, notably to replace the mutated ILR2G gene with the wild-type sequence of said gene.

A—Purifying CD34+ Hematopoietic Stem Cells (HSC) from Blood

Purification of CD34+ cells is well known to the person skilled in the art, and can be summarized as follows:
1—Donor blood samples (adult or umbilical cord blood rich in CD34+ cells) are collected, fresh or frozen (it is also possible to purify CD34+ cells from a patient with DISC-X);
2—Samples are centrifuged at 1000 g for 8 min without brake on sterile density gradient centrifugation medium (e.g. Ficoll in the presence of EDTA) to isolate Peripheral Blood Mononuclear Cells (PBMC);
3—The white blood cell rings are collected and washed with PBS (Phosphate Buffered Saline);
4—Hematopoietic stem cells (that is, CD34+ cells) are then isolated in a cell sorter by positive selection using anti-CD34 antibody labeling or by negative selection using a cocktail of anti-CD2, anti-CD3, anti-CD14, anti-CD16, anti-CD19, anti-CD24, anti-CD56n, anti-CD66b and anti-CD61 antibodies;
5—Purified CD34+ cells are centrifuged at 300 g for 5 min and the cell pellets are suspended at a cell density of 100,000 to 300,000 cells/mL and cultured at 37° C. and 5% CO₂ for 48 h in 24-well plates at a rate of 1 mL per well in a culture medium specifically dedicated to stem cell culture and supplemented with a cocktail containing human IL-3, human IL-6, recombinant thrombopoietin, recombinant hCSF (ligand Kit) and recombinant Flt-3 ligand, at 100 ng/ml each.
6—the CD34+ stem cells are thus ready to be transfected with the composition according to the invention.

### B—Preparing the Molecules of the Composition According to the Invention

To target the IL2R geneγ, and verify insertion at the IL2R locusγ chrX:70,327,254-70,331,958 GRCh37/hg19, a construct comprising GFP was prepared.

Two tubes were prepared as follows:
Tube 1 (10 μL):
oligo Great X1 (10 μM) corresponding to molecule 1 of sequence SEQ ID: 198 and on

5′-
AgaTgTGtatAAGAGaCAGGTAGTGTATTTTTTTTTTTTTTTTTATCAT
CCTGtCTCTTataCAcAtcTTTTTTTTTTTTTTTTTTTTTTTGGCGATC
GCTTTTTTTTTTTTTTTTGCTAGAAAGAGTACTGTTCTGGAAACTGACT
TTTTTTTTTTTTTTTGCGATCGCCTTTTTTTTTTTTTGATACATTTAga
TgTGtatAAGAGaCAGGATGAT-3′

oligo Great X3 (10 UM) corresponding to molecule 4 of sequence SEQ ID: 201 and on

5′-CACGTGCTGtCTCTTataCAcAtcTTTTCTCGATCATTATTATTTT
TTGGCGATCGCTTTTTTTTTTTTTTTTAGCAAATTGGTAATACTCCTGC
CTCCACAGTTTTTTTTTTTTTTTTGCGATCGCCTTTTTTTTTTTTTTTT
TTTTTAgaTgTGtatAAGAGaCAGCACGTGTTTTTTTTTTTTTTTTTTT
TCAGCTATACTGtCTCTTataCAcAtcT-3′

GREAT X1 Rev reverse oligo (10 μM) corresponding to the 5′ part of molecule 3 with sequence SEQ ID NO: 1134 and on

5′-
TACACTACCTGtCTCTTataCAcAtcTTTTTTTTTTTTTTTTTTTTTTT
ggttacATGCATCGTACA-3′

GREAT X3 Rev reverse oligo (10 μM) corresponding to the 3′ part of molecule 6 with sequence SEQ ID NO: 1135 and on

5′-
TGTACGATGCATgtaaccTTTTTTTTTTTTTTTTTTTTTTTAgaTgTGt
atAAGAGaCAGTATAGCTG-3′

the Reverse Helix Rev oligo corresponding to an additional strand for binding a helicase via the biotinylated 3′ end, the oligo having the sequence SEQ ID NO: 1136 and on

5′-ATAATAATGATCGAGAACTTTTTTTTTTTTTTTTTTTTTTTTTTTT
T[Biot]-3′

Tube 2 (10 μL):
oligo Great X2 (10 UM) corresponding to molecule 2 of sequence SEQ ID NO: 199 and on

5′-CACGTGCTGtCTCTTataCAcAtcTTTTCTCGATCATTATTATTTT
TTGGCGATCGCTTTTTTTTTTTTTTTTGTTGAGAATGGTGCTAGTGGTA
GTGAACAGTTTTTTTTTTTTTTTTGCGATCGCCTTTTTTTTTTTTTTTT
TTTTTAgaTgTGtatAAGAGaCAGCACGTGTTTTTTTTTTTTTTTTTTT
TGACGAATACTGtCTCTTataCAcAtcT-3′

oligo Great X4 (10 μM) corresponding to molecule 5 of sequence SEQ ID NO: 202 and on:

5′-
AgaTgTGtatAAGAGaCAGAGTATGAATTTTTTTTTTTTTTTTTATCAT
CCTGtCTCTTataCAcAtcTTTTTTTTTTTTTTTTTTTTTTTGGCGATC
GCTTTTTTTTTTTTTTTTCCTTCTCCTCTAAATCATTACCTTCTATAAT
TTTTTTTTTTTTTTTGCGATCGCCTTTTTTTTTTTTTGATACATTTAga
TgTGtatAAGAGaCAGGATGAT-3′

GREAT X2 Rev reverse oligo (10 μM) corresponding to the 3′ part of molecule 3 with sequence SEQ ID NO: 1137 and on

5′-CGATACgcggccgcatgttcTTTTTTTTTTTTTTTTTTTTTTTAga
TgTGtatAAGAGaCAGTATTCGTC-3′

GREAT X4 Rev reverse oligo (10 μM) corresponding to the 5′ part of molecule 6 with sequence SEQ ID NO: 1138 and on

5′-TTCATACTCTGtCTCTTataCAcAtcTTTTTTTTTTTTTTTTTgaa
catgcggccgcGTATCG-3′

the Reverse Helix Rev oligo corresponding to an additional strand for binding a helicase via the biotinylated 3′ end, the oligo having the sequence SEQ ID NO: 1136.

The two tubes are then heated to 95° C. for 5 min and then left for 1 hour at room temperature, then each tube is digested with the corresponding restriction enzymes: Tube 1: NsiI and Tube 2: NotI then purified by PCR purification kit (elution 20 μL).

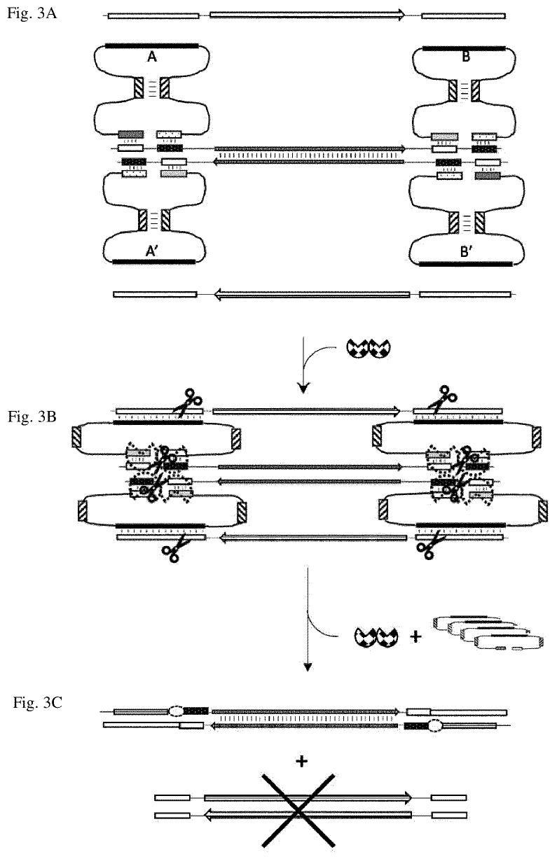

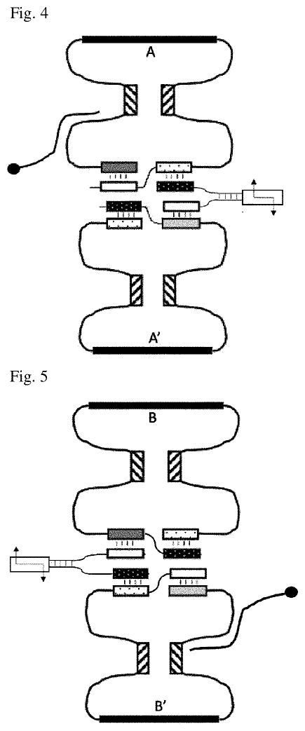

FIGS. 4 and 5 schematically show the contents of tubes 1 and 2 respectively before enzymatic digestion.

In parallel, an amplified GFP-ILR2γ sequence was obtained via lysis of HEK293T cells and genomic DNA extraction protocol and amplification of the locus of interest via hyper-processive Taq PCR. This sequence was amplified (using oligonucleotides with a 5′ NsiI restriction site and a 3′ NotI restriction site). The PCR product was digested by the two enzymes and purified by PCR purification kit eluted at a volume of 20 μL. The GFP-ILR2γ fusion is then flanked (that is, free NsiI and NotI half-sites) in tube 3.

The contents of the 3 tubes (tube 1, tube 2 and tube 3) are then mixed and T4 phage ligase is added to the mixture (4 μL, 100 U) in the presence of appropriate buffer (T4 buffer; 5 μL 10×) and 11 μL of distilled water. The mixture is left for 1 hour at room temperature. The mixture is then gel-purified (30 μL elution) to isolate the largest fragment, GFP-ILR2γ, with a size greater than 1 kbase.

The purified fragment is ready for use and includes the first, second, fourth and fifth molecules at the ends, the third and sixth corresponding to the GFP IL2Rγ fusion (tube 4).

At this stage, the modified single-stranded oligonucleotide complementary to part of the first and fourth molecules, with the 3′ part of the complementary modified single-stranded oligonucleotide coupled to biotin (biotinylated oligo), was added. This biotinylated molecule is not mandatory.

C—Transfection of CD34+ Cells with the Composition According to the Invention.

The contents of tube 4, containing biotinylated or non-biotinylated oligo, are then used to transfect isolated CD34+ cells by electroporation.

The cells are centrifuged at 300 g for 5 min and washed with PBS before being suspended in electrolyte buffer at a cell density of 75,000 to 100,000 cells per electroporation chamber. The contents of tube 4, together with a plasmid coding for the Tn5 protein, and optionally a plasmid coding a helicase-streptavidin fusion (UVRD-mSA), are added to the electroporation chamber according to the “Episomal iPSC Reprogramming Vectors” protocol of Thermofisher's Neon® Transfert System transfection system.

The mixture is electroporated at 1650 V in three 10 ms sequences.

The cells are then immediately centrifuged at 300 g for 5 min and suspended in supplemented stem cell culture medium for 48 to 96 h at 37° C. and 5% CO₂.

Tagmentation (that is, gene replacement) is then possible. The cells are maintained in a suitable culture medium.

### D—Transfection Verification

Transfected cells with the molecule of interest inserted at the locus are selected using an antibiotic whose resistance gene is contained in the replacement sequence (e.g. neomycin, bleomycin, blasticidin S etc.).

Genomic DNA from transfected and resistant cells is extracted and fragmented by sonication, then sequencing adapters are added. PCR amplification of all genomic DNA fragments is performed. This library was sequenced on a sequencer (50×8×50 reads on a HiSeq 2500, paired-end) and the results compiled by mapping and alignment via bwa-mem module (hg19 bank). On-target/off-target analysis was performed by specific selection and cluster read via comparison with reference genome (hg19) by mapping with the Bwa-mem module in short reads mode and the minimap2 module. Peak insertion data were assembled using the macs2 module.

It is also possible to perform a simple PCR using a sense oligonucleotide 5′ from the insertion and an antisense oligonucleotide 3′ from the insertion (Single-Tail Adapter/Tag (STAT)-PCR based method). The PCR product is then sequenced using conventional techniques, in order to detect in the region of interest at least the presence of GFP, a sign of fusion insertion.

### E—Functional Analysis of CD34+ Cells Modified by the Composition According to the Invention

Differentiation of modified CD34+ cells is monitored in vitro using the OP9-DL1 or DL4 murine stromal cell coculture model as described in the literature.

Briefly, OP9-DL1 or DL4 cells are cultured with modified CD34 cells for 14 days at 37° C. at 5% CO₂ and 10% O₂ in MEM (Minimum Essential Medium) supplemented with 10% SVF (Fetal Calf Serum), the cytokine cocktail required for CD34 cell culture (as described above) and a cytokine cocktail promoting activation of myelo-erythroid and lymphoid differentiation pathways.

After 2 weeks in culture, the differentiated CD34+ cells are collected and analyzed by flow cytometry to observe their hematopoietic differentiation profile using anti-CD19 (B lymphoid lineage) antibodies, anti-CD3 (T lymphoid lineage), anti-CD56 (NK lineage), anti-CD16 (monocyte/macrophage lineage), anti-CD14 (macrophage/neutrophil lineage), anti-CD11c (myeloid lineage) and anti-CD235a (erythroid lineage).

Differentiation of modified CD34+ cells is also monitored in vivo using the NSG or NOD SCID mouse model (immunodeficient mice).

Modified CD34+ cells are injected IH (intrahepatic) or IF (intrafemoral) into 3-4-day-old or 6-8-week-old mice.

Differentiation is monitored at 8 weeks, 12 weeks and 16 weeks post-injection, in the bone marrow, spleen and blood of mice. Human cells are analyzed by flow cytometry using anti-CD45 and anti-HLA ABC antibodies, and their cellular profile is analyzed using the same panel of antibodies as used in vitro.

For each cell type, GFP expression can be detected by flow cytometry.

For differentiated cells, reverse transcription can also be performed on isolated cells to obtain a cDNA library. Amplification of the GFP and ILR2Y sequence is then performed by PCR Oligos for PCR and IL2Rγ sequencing: ACTTGGCATATGOTTGTACAGCTCGTCCAT (SEQ ID NO: 1139) and GFP Rev: CACGAACTCCAGCAGGACCATG (SEQ ID NO: 1140) and the amplified fragment is sequenced by Sanger sequencing and migrated on agarose gel. As expected, after analysis (NCBI blast), we find a very close similarity with the GFP-IL2Rγ fragment theoretical replacement of Locus IL2Rγ.

These results show that the technology according to the invention enables the endogenous IL2Rγ gene to be efficiently replaced by an exogenous sequence.

Of course, the example given above is intended to demonstrate the effectiveness of the technology, and uses genes such as GFP to facilitate insertion detection.

In cell therapy, the gene coding for GFP is not used, and sequencing or functional detection of the receptor is used to verify efficacy.

### Example 2—Obtaining B Cells Expressing the Wild-Type ILR2G Gene from Pluripotent Stem Cells

Since it can be difficult to obtain CD34+ stem cells from the SCID-X patient, it is possible to use induced pluripotent stem cell (iPSc) technology.

IPSc cells can be derived from cell banks whose immunological profile is compatible with patients.

It is also possible to take differentiated cells from the patient (e.g. fibroblasts or epithelial cells), and force dedifferentiation by expressing the Oct4 and Socs2 genes, as well as other genes such as Klf4, Dub3, c-Myc, Nanog. Such techniques are now well known to the person skilled in the art, who will be able to adapt the protocols described in the prior art according to the cell type they wish to engage in a dedifferentiation model.

At this stage, iPSc cells can be transfected with lipofectamine in the presence of tube 4 as described in Example 1.

Transfected cells are selected using the appropriate antibiotic, and insertion is verified as described in Example 1.

IPSc cells are grown on cell matrix gel-coated plates, cocultured with inactivated murine embryonic fibroblasts for 24 h, in complete stem cell culture medium supplemented with bFGF and ROCK inhibitor.

The iPSc cells are then cultured for 7 days in 24 h culture media containing distinct differentiation cocktails each day:
D0-1: medium supplemented with hBMP-4, hVEGF, hWnt3a and KOSR;
D2: medium supplemented with hBMP-4, hVEGF, and KOSR;
D3: medium supplemented with hBMP-4, hVEGF, and bFGF;
D4-5: medium supplemented with hVEGF and bFGF;
D6: IMDM medium supplemented with F12, B27, N2, BSA, hVEGF, bFGF, human stem cell factor and hFlt3 ligand;
D7: IMDM medium supplemented with F12, B27, N2, BSA, hVEGF, bFGF, human stem cell factor, hFlt3 ligand, TPO, IL-6, hEPOgen, and FICZ (6-formylindolo[3,2-b]carbazole)

After 7 days, the cells are then maintained in culture for a further 3 to 7 days before continuing the experiments on the non-adherent cells harvested. These cells are tested for expression of the CD34+ marker by flow cytometry using an anti-CD34 antibody.

If the cells have not been transfected at the iPSc stage, they can then be transfected at the CD34+ stage as described in Example 1. Their differentiation is then induced as described in Example 1.

1-17. (canceled)

18. A composition comprising:
a) a first single-stranded nucleic acid molecule, comprising or consisting essentially of:
an A sequence allowing the insertion of a complementary sequence of a nucleic acid of interest, or comprising a complementary sequence of a nucleic acid of interest, said complementary sequence binding at the 5′ to a first T-rich sequence of 40 to 60 nucleotides in length and at the 3′ to a second T-rich sequence of 40 to 60 nucleotides in length, said first and second T-rich sequences respectively comprising a first and a second domain of 6 to 12 G/C-rich nucleotides, the sequence of the first domain being complementary to the sequence of the second domain, said first and second domains being positioned 15 to 52 nucleotides from said A sequence;
at least one first sequence oriented 5′-to-3′ for recognizing a transposase at the 5′ end of said first single-stranded nucleic acid molecule; and
at least one second sequence for recognizing said transposase at the 3′ end of said first single-stranded nucleic acid molecule,
b) a second single-stranded nucleic acid molecule, comprising or consisting essentially of:
a B sequence allowing the insertion of a complementary sequence of a nucleic acid of interest, or comprising a complementary sequence of a nucleic acid of interest, said complementary sequence binding at the 5′ to a third T-rich sequence of 40 to 60 nucleotides in length and at the 3′ to a fourth T-rich sequence of 40 to 60 nucleotides in length, said third and fourth T-rich sequences respectively comprising a third and a fourth domain of 6 to 12 G/C-rich nucleotides, the sequence of the third domain being complementary to the sequence of the fourth domain, said third and fourth domains being positioned 15 to 52 nucleotides from said B sequence;
at least one first sequence oriented 5′-to-3′ for recognizing said transposase at 5′ end of said second single-stranded nucleic acid molecule; and
at least one second sequence for recognizing said transposase at the 3′ end of said second single-stranded nucleic acid molecule,
said B sequence being a complementary sequence of said nucleic acid of interest, the A sequence being positioned at the 5′ of a region of interest of said nucleic acid of interest and the B sequence positioned at the 3′ of the region of interest of said nucleic acid of interest, and
c) a third single-stranded molecule, comprising or consisting essentially of:
at the 5′ end at least one complementary sequence of said second sequence for recognizing said transposase of the first molecule;
at the 3′ end at least one complementary sequence of said first sequence for recognizing said transposase of the second molecule; and
an intermediate region located between the complementary sequence of said second recognition sequence of said transposase of the first molecule and the complementary sequence of said first recognition sequence of said transposase of the second molecule, said intermediate region comprising a sequence coding for a portion of the interleukin-2 receptor gamma or IL2-Rγ, or coding for the complete IL2-Rγ receptor,
the first and third single-stranded nucleic acid molecules being paired according to the base complementarity defined by Watson and Crick so as to define two double-stranded binding sites of said transposase, and the second and third single-stranded nucleic acid molecules being paired according to the base complementarity defined by Watson and Crick so as to define two double-stranded binding sites of said transposase.

19. The composition according to claim 18, wherein said A sequence comprises a sequence complementary to the sequence of a first region of the gene coding for IL2-Rγ, and wherein said B sequence comprises a sequence complementary to the sequence of a second region of the gene coding for IL2-Rγ, said A sequence and said B sequence being two different sequences, said first and second regions of the gene coding for IL2-Rγ, flanking a region comprising a sequence coding for a portion of IL2-Rγ, or coding for the complete IL2-Rγ receptor.

20. The composition according to claim 18, wherein said first molecule comprises at the 5′ end a first sequence oriented 5′-to-3′ for recognizing a transposase and at the 3′ end a second sequence oriented 5′-to-3′ for recognizing said transposase, and wherein the third molecule comprises at the 5′ end a first complementary sequence of said first sequence for recognizing said transposase of the first molecule followed by a second complementary sequence of said second sequence for recognizing said transposase of the first molecule.

21. The composition according to claim 18, wherein said first molecule comprises at the 5′ end a first sequence oriented 5′-to-3′ for recognizing a transposase and at the 3′ end a second sequence for recognizing said transposase, followed by a first complementary sequence of said first sequence for recognizing said transposase, and
wherein the third molecule comprises at the 5′ end a complementary sequence of said second sequence for recognizing said transposase.

22. The composition according to claim 18, wherein said transposase is a bacterial transposase selected from the group consisting of Tn5, Tn9, Tn10, and Tc1/mariner.

23. The composition according to claim 18, wherein the gene coding for IL2-Rγ comprises a sequence selected from the group consisting of SEQ ID NO: 1, SEQ ID NO: 2, and SEQ ID NO: 3.

24. The composition according to claim 18, wherein the first, second and third molecules triplet is selected from the triplets as defined in Table 2.

25. The composition according to claim 18, further comprising:
d) a fourth single-stranded nucleic acid molecule, comprising or consisting essentially of:
an A′ sequence allowing the insertion of a complementary sequence of a nucleic acid of interest, or comprising a complementary sequence of a nucleic acid of interest, said complementary sequence binding at the 5′ to a fifth T-rich sequence of 40 to 60 nucleotides in length and at the 3′ to a sixth T-rich sequence of 40 to 60 nucleotides in length, said fifth and sixth T-rich sequences respectively comprising a fifth and a sixth domain of 6 to 12 G/C-rich nucleotides, the sequence of the fifth domain being complementary to the sequence of the sixth domain, said fifth and sixth domains being positioned 15 to 52 nucleotides from said A′ sequence;
at least one first sequence oriented 5′-to-3′ for recognizing a transposase at the 5′ end of said fourth single-stranded nucleic acid molecule; and
at least one second sequence for recognizing said transposase at the 3′ end of said fourth single-stranded nucleic acid molecule,
e) a fifth single-stranded nucleic acid molecule, comprising or consisting essentially of:
a B′ sequence allowing the insertion of a complementary sequence of a nucleic acid of interest, or comprising a complementary sequence of a nucleic acid of interest, said complementary B′ sequence binding at the 5′ to a seventh T-rich sequence of 40 to 60 nucleotides in length and at the 3′ to an eighth T-rich sequence of 40 to 60 nucleotides in length, said seventh and eighth T-rich sequences respectively comprising a third and a fourth domain of 6 to 12 G-C-rich nucleotides, the sequence of the seventh domain being complementary to the sequence of the eighth domain, said seventh and eighth domains being positioned 15 to 52 nucleotides from said B′ sequence,
at least one first sequence oriented 5′-to-3′ for recognizing a transposase at the 5′ end of said fifth single-stranded nucleic acid molecule; and
at least one second sequence for recognizing said transposase at the 3′ end of said fifth single-stranded nucleic acid molecule said B′ sequence being a complementary sequence of said nucleic acid of interest, the A′ sequence being positioned at the 5′ of a region of interest of said nucleic acid of interest and the B′ sequence positioned at the 3′ of the region of interest of said nucleic acid of interest, and
f) a sixth single-stranded molecule, comprising
at the 5′ end at least one complementary sequence of said fifth sequence for recognizing said transposase of the first molecule;
at the 3′ end at least one complementary sequence of said fourth sequence for recognizing said transposase of the second molecule; and
an intermediate region located between the complementary sequence of said fourth recognition sequence of said transposase of the first molecule and the complementary sequence of said fifth recognition sequence of said transposase of the second molecule, said intermediate region comprising a sequence that is antiparallel and complementary to the sequence coding for the interleukin-2 receptor gamma or IL2-Rγ contained in the third molecule of the first composition,
the fourth and sixth single-stranded nucleic acid molecules being paired according to the base complementarity defined by Watson and Crick so as to define two double-stranded binding sites of said transposase, and the fifth and sixth single-stranded nucleic acid molecules being paired according to the base complementarity defined by Watson and Crick so as to define two double-stranded binding sites of said transposase.

26. The composition according to claim 25, wherein
said A sequence comprises a sequence complementary to the sequence of a first region of the gene coding for IL2-Rγ,
said B sequence comprises a sequence complementary to the sequence of a second region of the gene coding for IL2-Rγ,
said A sequence and said B sequence being two different sequences, said first and second regions of the gene coding for IL2-Rγ, framing a region comprising a sequence coding for a portion of IL2-Rγ, or coding for the complete IL2-Rγ receptor,
said A′ sequence comprises a sequence complementary to the sequence of a third region of the gene coding for IL2-Rγ,
said B′ sequence comprises a sequence complementary to the sequence of a fourth region of the gene coding for IL2-Rγ,
said A′ sequence and said B′ sequence being two different sequences, said third and fourth regions of the gene coding for IL2-Rγ, framing a region comprising a sequence coding for a portion of IL2-Rγ, or coding for the complete IL2-Rγ receptor,
the A sequence and the A′ sequence are at most partially complementary,
the A sequence and the B′ sequence are at most partially complementary,
the B sequence and the A′ sequence are at most partially complementary, and
the B sequence and the B′ sequence are at most partially complementary.

27. A pharmaceutical composition comprising the composition according to claim 18 and a pharmaceutically acceptable carrier.

28. A method of replacing a mutated sequence of the gene coding for IL2-Rγ with a wild-type sequence coding for IL2-Rγ, said method comprising:
a) bringing the composition as defined in claim 18 into contact with the nucleic acid comprising the mutated sequence coding for the IL2-Rγ gene, wherein
the A sequence of said first molecule comprises a complementary sequence of the region immediately at the 5′ of the mutated sequence coding for the IL2-Rγ gene,
the B sequence of said second molecule comprises a complementary sequence of the region immediately at the 3′ of the mutated sequence coding for the IL2-Rγ gene, and
the third molecule comprises the wild-type sequence of the gene coding for IL2-Rγ, located between a complementary sequence of said second sequence for recognizing said transposase of the first molecule and the complementary sequence of said first sequence for recognizing said transposase of the second molecule to obtain a replacement complex,
b) placing the replacement complex in the presence of a transposase recognizing the double-stranded binding sites of said transposase contained in an ensemble, to obtain a recombination complex, and
c) recombining the combination complex to obtain the hybrid nucleic acid molecule the wild-type sequence of the gene coding for IL2-Rγ, in place of the mutated sequence of the gene coding for IL2-Rγ.

29. A method for the replacement of a mutated double-stranded sequence of the gene coding for IL2-Rγ of a stem cell, in particular a hematopoietic stem cell, by a wild-type double-stranded sequence coding for IL2-Rγ, said method comprising:
a) bringing a composition as defined in claim 18 into contact with the hematopoietic stem cell comprising the mutated sequence coding for the IL2-Rγ gene, wherein
the A sequence of said first molecule comprises a complementary sequence of the region immediately at the 5′ of the mutated sequence coding for the IL2-Rγ gene,
the A′ sequence of said fourth molecule comprises a complementary sequence of the region immediately at the 5′ of the mutated complementary sequence coding for the IL2-Rγ gene,
the B sequence of said second molecule comprises a complementary sequence of the region immediately at the 3′ of the mutated sequence coding for the IL2-R geneγ,
the B′ sequence of said second molecule comprises a complementary sequence of the region immediately at the 3′ of the mutated complementary sequence coding for the IL2-Rγ gene, and
the third molecule comprises the wild-type sequence of the gene coding for IL2-Rγ, located between a complementary sequence of said second sequence for recognizing said transposase of the first molecule and the complementary sequence of said first sequence for recognizing said transposase of the second molecule,
the sixth molecule comprises the wild-type sequence of the gene coding for IL2-Rγ, located between a complementary sequence of said second sequence for recognizing said transposase of the first molecule and the complementary sequence of said first sequence for recognizing said transposase of the second molecule to obtain a replacement complex,
b) placing the replacement complex in the presence of a transposase recognizing the double-stranded binding sites of said transposase contained in an ensemble, to obtain a recombination complex, and
c) recombining the combination complex to obtain the hybrid nucleic acid molecule the wild-type sequence of the gene coding for IL2-Rγ, in place of the mutated sequence of the gene coding for IL2-Rγ, and
d) possibly purifying hematopoietic stem cells with mutated gene replacement.

30. A method of inserting a double-stranded sequence of the wild-type gene coding for IL2-Rγ into a cell, in particular a stem cell, in particular a hematopoietic stem cell, at the locus of the gene coding for IL2-Rγ, wherein said locus of the gene coding for IL2-Rγ comprises a mutation affecting the expression, the function, or both, of IL2-Rγ, and wherein said double-stranded sequence of the wild-type gene coding for IL2-Rγ corresponds to the cDNA of said gene, comprising:
a) brining a composition according to claim 18 into contact with the cell comprising a mutation affecting the expression, function or both of IL2-Rγ, at the locus of the gene coding for IL2-Rγ, wherein
the A sequence of said first molecule comprises a complementary sequence of the region at 5′ of exon 1 of the gene coding for IL2-Rγ,
the A′ sequence of said fourth molecule comprises a complementary sequence of the complementary sequence of the region at 5′ of exon 1 of the gene coding for IL2-Rγ,
the B sequence of said second molecule comprises a complementary sequence at 3′ of the region at 5′ of exon 1 of the gene coding for IL2-Rγ,
the B′ sequence of said fifth molecule comprises a complementary sequence at 3′ of the complementary sequence of the region at 5′ of exon 1 of the gene coding for IL2-Rγ, and
the third molecule comprises the wild-type cDNA sequence of the gene coding for IL2-Rγ, located between a complementary sequence of said second sequence for recognizing said transposase of the first molecule and the complementary sequence of said first sequence for recognizing said transposase of the second molecule,
the sixth molecule comprises the wild-type cDNA sequence of the gene coding for IL2-Rγ, located between a complementary sequence of said second sequence for recognizing said transposase of the first molecule and the complementary sequence of said first sequence for recognizing said transposase of the second molecule to obtain a replacement complex,
b) placing the replacement complex in the presence of a transposase recognizing the double-stranded binding sites of said transposase contained in an ensemble, to obtain a recombination complex, and
c) recombining the combination complex to obtain insertion of the cDNA of the gene coding for IL2-Rγ 5′ to exon 1 of the gene coding for IL2-Rγ, and
d) if necessary, purifying the inserted cell.
